(19) United States
(12) Patent Application Publication
(10) Pub. No.: US 2014/0322356 A1
(43) Pub. Date: Oct. 30, 2014

(54) CTC BIOMARKER ASSAY TO COMBAT BREAST CANCER BRAIN METASTASIS

(71) Applicant: Baylor College of Medicine, Houston, TX (US)

(72) Inventors: Dario Marchetti, Seabrook, TX (US); Lon Ridgway, Houston, TX (US); Lixin Zhang, Houston, TX (US)

(73) Assignee: BAYLOR COLLEGE OF MEDICINE, Houston, TX (US)

(21) Appl. No.: 14/361,248

(22) PCT Filed: Nov. 28, 2012
(86) PCT No.: PCT/US12/66868

§ 371 Date: May 28, 2014

Related U.S. Application Data
(60) Provisional application No. 61/563,959, filed on Nov. 28, 2011.

(51) Int. Cl. C12Q 1/68; G01N 33/574

(52) U.S. Cl. CPC C12Q 1/6886; G01N 33/57415; C12Q 2600/158; C12Q 2600/118; G01N 2333/705; G01N 2800/50; G01N 2800/60
USPC 424/649; 435/7.23; 435/6.11; 435/6.12

(57) ABSTRACT

Embodiments of the present invention concern methods related to treating, prognosticating and/or diagnosing at least brain metastatic breast cancer. Embodiments of the methods include characterizing circulating tumor cells for the presence or absence of EpCAM and, upon identification of EpCAM negative cells and identification of the status of other markers (such as heparanase and/or Notch1, for example), treating the individual based on the determination of the characterization.

### CROSS-REFERENCE TO RELATION APPLICATIONS

This application claims priority to Provisional Patent Application Ser. No. 61/563,959 filed Nov. 28, 2011, which application is incorporated by reference herein in its entirety.

### STATEMENT REGARDING FEDERALLY SPONSORED RESEARCH OR DEVELOPMENT

This invention was made with government support under R01 CA 1610335 awarded by National Institutes of health. The government has certain rights in the invention.

### TECHNICAL FIELD

The fields of the invention include at least cell biology, molecular biology, medicine, and diagnostics, including of breast cancer, such as brain metastatic breast cancer.

### BACKGROUND OF THE INVENTION

The overwhelming majority of cancer deaths are due to metastasis (Talmadge and Fidler, 2010). Among them, brain metastatic breast cancer (BMBC) represents the most feared consequence of breast cancer since uniformly fatal, increasing in frequency, with occult brain metastasis being exceptionally common at autopsy (Eichler et al., 2011). Circulating tumor cells (CTCs) represent the primary cause of intractable metastatic disease and are considered essential for metastasis formation (Cristofanilli et al., 2004: Pantel et al., 2008; Pantel et al., 2011). However, characterization of CTCs inductive of metastasis remains elusive since a variety of platforms are unable to capture the entire spectrum of CTCs due to their phenotypic heterogeneity and the complexities of events within the metastatic cascade (Eichler et al., 2011; Cristofanilli et al., 2004 Pantel et al., 2008; Pantel et al., 2011). For example, the CellSearch™ platform, the only CTC prognostic test approved by the US Federal and Drug Administration (FDA), relies on the use of antibodies targeting the epithelial cell adhesion molecule (EpCAM), thus it is only capable of capturing EpCAM-positive CTCs, but neither EpCAM—undetectable or EpCAM-negative (both termed “EpCAM-”) CTCs. Multiple studies have also demonstrated that CellSearch™ is unable to capture CTCs in 30-35% of metastatic breast cancer patients (Cristofanilli et al., 2004: Pantel et al., 2008; Pantel et al., 2011), while over 60% of patients with BMBC have undetectable CTCs by CellSearch™ analyses (Pantel et al., 2011). Therefore, new approaches to identify and characterize EpCAM-CTCs in breast cancer patients are needed. They are critical to improve mechanistic understandings of BMBC biology, why and how metastasis occurs, with the ultimate objective to develop novel and more efficacious treatments to improve patient's survival. The objective of current study was to develop approaches to detect, isolate, and characterize EpCAM-negative CTCs present in breast cancer patients and interrogate their metastatic competence to brain. Many studies have shown that methods for CTC detection, isolation, and enrichment are based on density gradient centrifugation and immunomagnetic procedures (Pantel et al., 2008; Pantel et al. 2011; Stott et al., 2010; Nagrath et al., 2007; Pecot et al., 2011). However, they can be limiting because of the heterogeneous nature of CTCs and inabilities to investigate EpCAM-CTCs (Sieuwerts et al., 2009; Königsberg et al., 2011). Further, accumulated evidence has demonstrated that only a very small number of CTCs survives in the circulation and possesses all the properties to generate distant metastasis (Talmadge and Fidler, 2010: Eichler et al. 2011). This fraction is represented by CTCs that may have lost EpCAM expression and believed to have undergone the process of epithelial mesenchymal transition (EMT) which results in a spectrum of epithelial cell surface antigens shedding and the downregulation of epithelial CTC markers, e.g., E-cadherin, claudins, and cytokeratins (Königsberg et al., 2011: Harrell et al., 2012; Joosse et al., 2012; Mego et al., 2010). Several groups have also reported that CTCs express stem cell and/or EMT-associated markers (Pecot et al., 2011; Sieuwerts et al., 2009; Mego et al., 2010); however, it is unclear whether CTCs that no longer express EpCAM, thus evading detection by the CellSearch™ platform, are metastasis-competent. No direct proof demonstrating that CTCs captured from blood of cancer patients are seeds for tumors has been presented thus far. As mentioned in a recent Science article (Kaiser, 2010) “A few labs are going a step further, trying to directly show that human CTCs can cause new tumor”, investigators aim to isolate and culture CTCs from clinical patients, then inject them in xenografts; and evaluate whether metastases are generated in these animals. Although murine CTCs have been successfully cultured (Ameri et al. 2010; Maheswaran et al., 2008), no long-term in vitro growth for human CTCs derived from cancer patients—establishing CTC lines has been reported in the literature. Similarly, assessing metastatic competency and biomarkers in BMBC patients' CTCs has not been reported. In the present invention, the inventors provide first-time evidence demonstrating the identification, growth, and characterization of CTCs from cancer patients. Second, the inventors prove metastatic competency of these CTCs once they were injected in immunodeficient animals. Lastly, the inventors show that a set of biomarkers present in CTCs is necessary to generate BMBC: the CTC signature.

### BRIEF SUMMARY OF THE INVENTION

The present invention is directed to a system, method, and/or compositions for characterizing samples from individuals for brain metastatic breast cancer (BMBC). In specific aspects to the invention, the methods are utilized to be able to predict and guide treatment for BMBC.

In some embodiments of the invention, there is identification and characterization of breast cancer CTCs competent for brain metastasis. In particular embodiments of the invention, there is provided a CTC biomarker assay to identify or characterize breast cancer brain metastasis. In certain aspects of the invention, there are subsets and signatures of breast cancer brain-homing circulating tumor cells, and embodiments of the invention allow their isolation and/or characterization. In specific embodiments, such information is utilized in determining a treatment regimen for BMBC or breast cancer or prevention of BMBC or prevention of breast cancer, for example. Such embodiments include more frequent and/or intense monitoring of the individual for the presence of breast cancer or its metastasis.

The present invention addresses deficiencies in the prior art by identifying a novel marker set of genes that are differentially expressed in particular cells for the prognosis and/or diagnosis of at least breast cancer brain metastasis, including an indication that an individual requires a certain treatment regimen when the individual has particular expression patterns of certain genes referred to herein. The encoded mRNA species (and/or the corresponding encoded protein species, in at least some cases) from these gene(s) have utility, for example, as markers of BMBC cancer. Antibodies against the encoded protein species, as well as antisense constructs specific for particular mRNA species, have utility for methods of therapeutic treatment of BMBC (including for heparanase and Notch1). In addition, the corresponding respective DNA sequences of the signature can be used to design probes and primers, for example.

The nucleic acid sequence for the specific genes can be used to design specific oligonucleotide probes and primers. When used in combination with nucleic acid hybridization and amplification procedures, these probes and primers permit the rapid analysis of specimens, liquid samples, including blood or serum samples, etc. This assists physicians in diagnosing BCBM or prognosticating BCBM to allow determination of optimal treatment courses for individuals with BCBM. The same probes and primers also may be used for in situ hybridization or in situ PCR detection and diagnosis of BCBM, for example.

In one embodiment of the present invention, the isolated nucleic acids of the present invention are incorporated into expression vectors and expressed as the encoded proteins or peptides. Such proteins or peptides may in certain embodiments be used as antigens for induction of monoclonal or polyclonal antibody production.

One aspect of the present invention includes oligonucleotide hybridization probes and primers that hybridize selectively to BCBM samples or samples suspected of comprising BCBM. The availability of probes and primers specific for such BCBM specific nucleic acid sequences, that are differentially expressed in BCBM, provides the basis for diagnostic kits useful for distinguishing between those individuals having a risk of or susceptibility for developing BCBM and those that do not.

In one aspect, the present invention encompasses methods and/or kits for use in characterizing BCBM cancer cells in a biological sample wherein there may be cells that are EpCAM negative and that optionally comprise expression of heparanase (HPSE) and/or Notch1. Such a kit may comprise one or more pairs of primers for amplifying nucleic acids corresponding to EpCAM, HPSE, Notch1, HER2/neu; EGFR; uPAR; ALDH1; cytokeratins; CD44^{high}/CD24^{low}; vimentin; and/or CD45.

The kit may further comprise samples of total mRNA derived from tissue of various physiological states, such as normal, breast cancer, and/or metastasized breast cancer for example, to be used as controls. The kit also may comprise buffers, nucleotide bases, and other compositions to be used in hybridization and/or amplification reactions. Each solution or composition may be contained in a vial or bottle and all vials held in close confinement in a box for commercial sale. Another embodiment of the present invention encompasses a kit for use in detecting BCBM cells in a biological sample comprising oligonucleotide probes effective to bind with high affinity to nucleic acids corresponding to the one or more respective genes in a Northern blot assay and containers for each of these probes. In a further embodiment, the invention encompasses a kit for use in detecting BCBM in a biological sample comprising antibodies specific for the corresponding proteins identified in the present invention.

In one broad aspect, the present invention encompasses methods for treating BCBM patients by administration of effective amounts of antibodies specific for certain peptide products of the signature, and/or by administration of effective amounts of vectors producing antisense messenger RNAs, for example, that bind to certain nucleic acids corresponding to the signature, and/or by any therapy useful in treating and/or alleviating at least one symptom of BCBM. Antisense nucleic acid molecules also may be provided as RNAs, as some stable forms of RNA with a long half-life that may be administered directly without the use of a vector are now known in the art. In some cases appropriate siRNA or miRNA molecules are employed. In addition, DNA constructs may be delivered to cells by liposomes, receptor mediated transfection and other methods known in the art. miRNAs may be employed for therapeutic embodiments. Delivery of the present agents, by any means known in the art would be encompassed by the present claims.

The invention further comprises methods for detecting BCBM cells in biological samples, using hybridization primers and probes designed to specifically hybridize to nucleic acids corresponding to one or more particular genes of the signature. This method further comprises identification of the absence or presence or measuring the amounts of nucleic acid amplification products formed when primers selected from the designated sequences are used.

The invention further comprises the prognosis and/or diagnosis of BMBC by identification of the absence or presence or measuring the amounts of nucleic acid amplification products formed as above. The invention comprises methods of treating individuals with BCBM by providing effective amounts of antibodies and/or antisense DNA molecules that bind to particular of the products of the above mentioned isolated nucleic acids. The invention further comprises kits for performing the above-mentioned procedures, containing antibodies, amplification primers and/or hybridization probes, for example.

The invention further comprises therapeutic treatment of breast cancer, including BMBC, by administration of effective doses of inhibitors specific for the aforementioned encoded proteins when they are upregulated.

In embodiments of the invention, an individual that is subjected to method(s) of the invention is an individual that is suspected of having, known to have, or at risk of having breast cancer, including all types of breast cancer, such as brain metastatic breast cancer. The method(s) may be performed at the initial diagnosis of breast cancer or during a routine screening for an individual, or the individual may already have or be at risk for metastatic breast cancer.

In specific embodiments of the invention, the breast cancer of the individual may be estrogen receptor (ER) positive or negative, although in particular cases it is ER negative. In specific embodiments of the invention, the breast cancer of the individual may be progesterone receptor (PR) positive or negative, although in particular cases it is PR negative. In some aspects of the invention, the cancer cells have overexpression of EGFR1, EGFR2, or HER2.

In some embodiments, an individual is subjected to one or more diagnostic methods for BMBC in addition to the diagnostic embodiments of the invention. In specific embodiments, some methods are employed, such as magnetic resonance imaging, CAT scan, and so forth.

In some embodiments of the invention, the methods of the invention are utilized in conjunction with other CTC analysis procedures, such as CellSearch™.

In specific embodiments of the invention, the expression levels and/or patterns (such as subcellular localization) of one or more of the members of the gene signature are identified.

In one embodiment of the invention, there is a method of identifying the presence of or risk for brain metastatic breast cancer in an individual, comprising the step of identifying from a sample from the individual circulating cells that are epithelial cell adhesion molecule (EpCAM) negative and that comprise expression of heparanase (HPSE) and/or Notch1. In a specific embodiment, the cells further comprise one or more of the following markers: a) HER2/neu; b) EGFR; c) uPAR: d) ALDH1; e) cytokeratins; f) CD44high/CD24low; g) vimentin; and h) CD45. In a specific embodiment, the cells are circulating tumor cells (CTCs) are peripheral blood mononuclear cells. In a specific embodiment, the HPSE is localized to the nucleus or nucleolus of cells from the CTCs from the sample.

In a specific embodiments, the presence of the markers is determined by immunofluorescence, fluorescence in situ hybridization, flow cytometry, polymerase chain reaction, or a combination thereof. In some embodiments, the method is employed in conjunction with another method for identifying brain metastatic breast cancer or breast cancer in an individual.

In some embodiments of the invention, there is a method of identifying the presence of or risk for brain metastatic breast cancer in an individual, comprising the step of identifying from a sample from the individual circulating cells that are epithelial cell adhesion molecule (EpCAM) negative.

In some embodiments of the invention, there is a method of treating an individual for brain metastatic breast cancer or delaying the onset of brain metastatic breast cancer in an individual, or preventing brain metastatic breast cancer in an individual or preventing metastasis of breast cancer in an individual or preventing breast cancer, comprising the step of providing an effective amount of a therapy to the individual when the individual has had identified the presence of circulating cells that are epithelial cell adhesion molecule (EpCAM) negative and that comprise expression of heparanase (HPSE) and/or Notch1 in a sample from the individual. In specific embodiments, the therapy is selected from the group consisting of surgery, radiation, immunotherapy, chemotherapy, hormone therapy, steroids, and a combination thereof. In some embodiments, the cells further comprise one or more of the following markers: a) HER2/neu; b) EGFR; c) uPAR; d) ALDH1; e) cytokeratins; f) CD44high/CD24low: g) vimentin; and h) CD45.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows representative flow cytometry of ALDH1, CD45, EpCAM and HPSE of peripheral blood mononuclear cells isolated from patients with BMBC. Cells were first sorted for CD45, ALDH, then for EpCAM status obtaining EpCAM positive and EpCAM negative CTC subsets. Red box indicates the number of EpCAM⁻/CD45⁻/ALDH1⁺ and EpCAM⁺/CD45⁻/ALDH1⁺ CTCs recovered from FACS of PBMCs from a BMBC patient. Cells were subsequently collected and grow in vitro. Images of CTCs by phase contrast microscopy and HPSE expression (green fluorescence signal) by confocal microscopy and cytospins are shown (left and right panels). Of note, the EpCAM⁺/CD45⁺/ALDH1⁻ status of FACS-sorted CTCs subsets were confirmed by CellSearch™ analyses. The visualization of CTCs defined in EpCAM⁺/CD45⁺/ALDH1⁻-nucleated (DAPI+) cells is shown (right panels).

FIG. 2 shows gene expression of FACS-selected CTCs from BMBC patients (Pt. A-C) compared to patient PBMCs analyses after Ficoll-Hypaque but before FACS isolation (Pt. D) or control PBMCs from healthy donors (normal). Square delineates a common CTC signature.

FIG. 3 shows brain metastatic competency of CTCs isolated from blood of BMBC patients. CTCs possessing the BMBC CTC signature and cultured in vitro were infected I Scid mice and metastasis monitored. Multiple brain micro-metastasis surrounded by neuroglial tissue were detected in these animals (circles). Insert shows BMBC tissue from the same patient whose blood was analyzed for BMBC-competent CTCs.

FIG. 4 demonstrates EpCAM-negative/Notch-1 overexpressor CTCs. Displays (A, B, C) represent FACS analyses for distinct CTC lies obtained from three BMBC cases. (Top row) Portion of total viable population selected for sorting. (Middle row) FAS of cells without Notch-1-APC or EpCAM-PE fluorescence-conjugated primary antibodies. (Bottom row) FACS of Notch-1-APC (+) and EpCAM-PE (−) populations detected using fluorescence-conjugated primary antibodies. Positive and negative controls for EpCAM and Notch1 were performed to assess signal specificity; in addition to monitoring fluorescence within selected wavelengths. Percentages of EpCAM-negative/Notch-1 overexpressors from each sorted population are indicated.

FIG. 5 shows ALDH1 activity in PBMCs from BMBC patients. Representative FACS analyses of PBMC from a BMBC patient for ALDH activity by the ALDEFLUOR assay. Cells were incubated with ALDEFLUOR substrate (BAAA) and the specific inhibitor of ALDH1. DEAB, to establish the baseline fuorescence and to define the ALDEFLUOR-positive region. Incubation of cells with ALDEFLUOR substrate in the absence of DEAB induces a shift in BAAA fluorescence defined in the ALDEFLUOR-positive population.

FIG. 6 shows EGFR gene amplification correlates with nuclear HPSE expression in brain metastatic breast cancer (BMBC) patient blood. A. Representative images of FISH analyses for EGFR gene amplification in CTCs isolated from BMBC patient blood. A custom made probe which contains LSIEGFR 7p12, CEP10 and 10q probes (Cytocell/Rainbow Scientific Inc., Windsor, Conn.) was used for FISH analysis. A significant EGFR gene amplification was detected (spectrum green, arrows), compared to CEP10/10q copies number (acqua and red color, respectively). DAPI indicates nuclear staining (blue). B. FISH and IF analyses correlating EGFR gene amplification with heparanase (HPSE) expression. A LSI EGFR/Cep7 probe (Abbott Molecular Inc., Chicago, Ill.) was used for FISH assay. LSI EGFR 7p12 and centromeric 7 (ploidy content) were labeled with spectrum orange and acqua color, respectively. DAPI indicates nuclear staining (blue). IF analyses for HPSE were performed using a monoclonal anti-HPSE antibody and completed before FISH. Extensive intranuclear HPSE presence is detected (green fluorescence). C. Representative images of IF analyses showing a correlation of HPSE and the tumor-initiating cell (cancer stem cell) marker aldehyde dehydrogenase 1 (ALDH1) in nucleated CTCs from blood of BMBC patients. Immunofluorescence for BMBC peripheral blood mononuclear cells (PBMCs), MDA-MB-231BR, and PBMCs from patient without breast cancer for DAPI (nuclear staining), HPSE. ALDH1, and HPSE/ALDH1 combinations (Merge). MDA-231 BR and control PBMCs were used for ALDH1 positive and negative controls, respectively. Representative analyses of HPSE/ALDH1 patterns in EGFR gene-amplified CTCs from BMBC patients. *PBMCs from patients without breast cancer. **A total of 3.0×10⁶ PBMCs isolated from metastatic breast cancer patients were loaded on FICTION/BioView™ system for marker analysis. The system randomly scanned approximately 5.0×10³ cells for the each marker/sample.

FIG. 7. CTC identification and culture. Primary sorting of PBMCs obtained from Ficoll-Hypaque gradients were completed using selection markers (ALDH1, EpCAM and CD45). Based on EpCAM positivity, cells were divided into two groups: EpCAM+/ALDH1+/CD45−, and EpCAM−/ALDH1+/CD45+. Cells were collected under sterile conditions and cultured using specific culture procedure as described in Materials and Methods. A. Cells were monitored for growth daily and representative images at indicated days were shown for cell morphology. Magnification 200×. B. Representative images of IF analyses of specific cytokeratin (CK5/6/18), ALDH1, EpCAM, and vimentin in the EpCAM−/ALDH1+/CD45− and DAPI-positive cells. Magnification 400×. C. Cytokeratin 16 (CK16) was analyzed by Western blot analysis, since expression level of CK16 is critical in the detection of metastatic breast cancer CTCs (Joosse et al., 2012). AE1 antibody (Millipore, Cat #MAB1612) recognizes CK4, CK6 and CK9 (Joosse et al., 2012). MDA-MB-231 parental and the brain-metastatic variant (MDA-MB-231BR) cells were used as positive controls. β-actin was used as control for equal loading. D. RT-PCR analyses of CTCs. Selected genes were classified into four groups based on the function as indicated. The MDA-MB-231BR line was used for control of metastatic breast cancer. GAPDH was used for loading control. PBMCs derived from patients with or without breast cancers were used as additional controls of CTC signature specificity and sensitivity. E. RT-PCR analyses of CTCs lines following 20 passages of in vitro culture. F. RT-PCR analysis of CTCs for non-CTC specific markers (Dominici et al., 2006; Fonsatti et al., 2000: Ostapkowicz et al., 2006; Bos et al., 2009). G. Expression of the signature proteins in CTCs was examined by IF. Graph shows signal quantification of positive staining of signature proteins. The expression of vimentin was also quantified.

FIG. 8. Sorting and characterization of CTCs over-expressing Notch1, EGFR, and HER2. A. FACS analysis and capture of viable CTCs by EpCAM-negative, Notch1 over-expressors (top). Captured cells were expanded in tissue culture and further sorted for EGFR and HER2 over-expression (bottom). The percentage of positive cells for the each sorting is indicated. MCF-7 and SK-BR-3 cells were used as positive controls. B. Immunofluorescence analysis of the expression levels of the signature proteins (EGFR, HER2, Notch1, HPSE, and EpCAM) in FACS-sorted CTC over-expressors. Insert shows EpCAM staining in breast cancer ZR75 cells (luminal sub-type: (Sieuwerts et al., 2009; Königsberg et al., 2011) as EpCAM-positive cell line (control). C. HPSE activity (HS degrading assay, Takara Inc., Takarazuka, Japan) was examined in CTC over-expressors. MCF-7 and MDA-MB-231BR cells were used as negative and positive controls, respectively (Zhang, Sullivan, et al., 2001).

FIG. 9. CTC invasion and experimental metastasis assays. A. CTCs possess high invasive capabilities. Chemoinvasion analyses were performed using Matrigel™ chemoinvasion chambers. Top: Representative images of chamber inserts are shown. Bottom: Cell invasion values were quantified per experiment. Poorly invasive MCF-7 and highly invasive MDA-MB-231BR breast cancer cells were used as controls. Bars represent the standard deviation of the mean of 8-10 fields/cell line/assay. B. Lung metastatic competency mediated by CTC over-expressors. Mitoses in lung metastasis are indicated by arrows. C. CTCs-induced breast cancer brain metastasis. Representative images show that multiple brain micro- and macro-metastasis surrounded by neuroglial tissue were mediated by CTCs over-expressing the signature proteins. Aberrant mitosis (arrows) were observed. D. Representative images and quantification of CTC-ov mediated brain metastasis in mouse model. Top. Hematoxilin & Eosin (H&E) staining sections showing CTC-induced breast cancer brain metastasis in a mouse model. Bottom. Representative images of brain metastasis and specific quantification of tumor cells by the Cri Vectra-Inform™ Intelligent imaging analysis system was selected from corresponding H&E sections (Cambridge Research & Instrumentation, Inc., Boston, Mass.). The Inform™ software is based on equipment-learning program that can be trained to generate specific tumor cell quantifications by drawing around tumor images. The software recognizes and distinguishes significant histological features including tumor or no tumor. Eight H&E tissue sections were selected from mice sub27 groups with BMBC induced by CTC over-expressors. Graphs show the quantification of tumor cells defined in representative mouse brains. E. Expression of CTC signature proteins in a mouse brain. Animals were injected with CTCs over-expressing the signature proteins. Brain metastasis was confirmed by H&E and pathological assessment, and proteins of the CTC signature were examined by immunohistochemistry. Shown are representative results from experiments performed in quadruplicate.

FIG. 10. CTCs cell morphology. The three CTC lines established from respective patients were stained using the Diff-Qick stain (Kaiser, 2010), and cell morphology was examined under microscopy.

FIG. 11. Cells sorted from BMBC patients were spiked into 7.5 mls of blood from healthy donors and analyzed by CellSearch™ (Cristofanilli et al., 2004; Pantel et al., 2008). Each of the above CTC lines was spiked in a dose-dependent manner in five independent experiments/CTC line/CellSearch™ analysis. Right panel. Representative CellSearch™-captured EpCAM+ CTCs with EGFR positivity assessed in parallel using the fourth fluorescence channel of CellSearch™. Human breast cancer SK-BR-3 cells were used as a positive control of EpCAM expression being an integral component of the CellSearch™ control CTC kit (Fehm et al., 2010). A representative image of EGFR+ CTC visualized by CellSearch™ is displayed (bottom).

FIG. 12. Selective EGFR immunoreactivity in CTC-induced breast cancer brain metastasis. A. Murine BMBC. B. Patient BMBC.

FIG. 13. CTC metastatic competency: CTCs induced lung tumors in animals show similar cell morphology to the original BMBC tissue from patients whose blood was analyzed for CTCs.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein.

### I. General Embodiments of the Invention

The identification and characterization of circulating tumor cells (CTCs) causing fatal metastases remain elusive. Metastatic disease is incurable, thus new approaches to predict and prevent the development of metastases are needed. Drug combinations are infrequently tested for their effectiveness in preventing metastatic colonization. Thus, the inhibition of organ-specific metastasis using targeted therapies could be better investigated if coupled with CTC-associated characteristics, predicting metastasis to the organ of interest. For example, the incidence of breast cancer brain metastasis (BCBM) appears to be increasing. BCBM is particularly common in patients whose tumors are negative for estrogen/progesterone receptors and possess an over-expression of epidermal growth factor receptor1 or 2 (EGFR or HER2). However, investigations using therapies targeting HER2/EGFR showed only limited success in the clinical management of BCBM. The inventors considered that profiling CTCs from patients with BCBM would result in the identification of brain-colonizing CTC signatures with clinical utility.

To this end, the inventors used (as an example) fluorescence-activated cell sorting (FACS), RT-PCR employing novel oligo sequences, CellSearch™ and a technology analyzing antigenic markers by immunofluorescence, coupled with detecting gene amplification by fluorescence in situ hybridization on the same cells; and quantification of the signal via automated scanning (FICTION; BioView Duet-3™ system). The inventors established the feasibility of the approaches by performing CTC analyses on peripheral blood mononuclear cells isolated from BCBM patients or patients not possessing overt brain metastatic disease. From these patient samples, the inventors: 1) detected a differential gene amplification for EGFR and HER2; 2) found that the number of CTCs visualized by the BioView™ platform was at least three orders of magnitude higher than the number obtained from CellSearch™ from the same specimen; 3) identified a significant correlation between the presence of BCBM and CTCs not detectable by CellSearch™ (CellSearch only identifies EpCAM-positive CTCs). Conversely, these CTCs contained high levels of pro-metastatic heparanase, in conjunction with the expression of aldehyde dehydrogenase-1 (ALDH-1), a known cancer stein-cell marker, and with high correlation between heparanase, ALDH-1, and EGFR gene amplification. Further, by using combinatorial flow cytometric/FACS analyses, the inventors demonstrated the presence of CTC subsets negative for EpCAM and CD45 (a hematolymphoid marker), however enriched for heparanase/ALDH-1 expression; established procedures for retrieving viable FACS-derived CTC subsets amenable to growth in vitro; and discovered a specific association in CTC subset profiling of HER-2, EGFR, CD44^{high}/CD24^{low}, Notch1, and Heparanase gene expression, consistent with: i) EpCAM negativity; ii) superior Notch1 expression over ALDH-1 as marker of the stem cell pool; iii) a correlation with the onset of BCBM in patients and in highly immunodeficient mice (xenotransplantation studies). The characterization of (TC subtypes in patients with BCBM indicate the discovery of BCBM founder CTCs. One can characterize properties of CTC subtypes in their abilities for metastatic competency and organ homing specificity, notably to brain using routine methods in the art.

The present invention includes embodiments wherein CTCs from an individual suspected of having BMBC or at risk for having BMBC or suspected of having breast cancer or at risk for having breast cancer are evaluated for the presence of one or more gene markers, and a treatment regimen and/or monitoring regimen is implemented upon such a determination. Such monitoring may include routine or non-routine methods to evaluate the individual's breast health, and such monitoring may occur at the same or increased frequency compared to an individual that is not known to have breast cancer or not known to have BMBC or not suspected of same.

Upon determination of the presence of the particular CTCs described herein, an individual that is not known to have breast cancer may be provided at an earlier age and/or with increased frequency a monitoring regimen to ascertain the onset of breast cancer. Upon determination of the presence of the particular CTCs described herein, an individual that is known to have breast cancer may be provided with a monitoring regimen to ascertain whether or not there is metastasis and/or may be subjected to preventative or therapeutic measures to avoid the onset or delay the onset of metastasis. Such a monitoring regimen may be at an increased frequency in the individual over an individual not known to have the particular CC/gene signature.

### II. Treatment of BMBC

In certain embodiments of the invention, an individual is treated for BMBC upon the useful information provided in particular embodiments of the invention. In specific aspects, an individual is provided treatment for BMBC and/or one or more symptoms thereof and/or palliative treatment when an individual is recognized as having, for example, circulating cells (including circulating tumor cells) that are epithelial cell adhesion molecule (EpCAM) negative and that comprise expression of heparanase (HPSE) and/or Notch1. In some cases, the cells further comprise one or more of the following markers: a) HER2/neu; b) EGFR; c) uPAR; d) ALDH1; e) cytokeratins; f) CD44^{high}/CD24_{low}; g) vimentin: and h) CD45.

Treatment for the BMBC may comprise one or more of steroids, anti-seizure medication, whole-brain radiation, surgical excision, stereotactic radiotherapy, adjuvant radiation, radiosensitization, chemotherapy (Xeloda (capecitabine), high-dose mexthotrexate, the platinum drugs carboplatin and cisplatin, and Adriamycin (doxorubicin), lapatinib, and combinations thereof), and hormone therapy (tamoxifen, letrozole (Femara) and/or megestrol acetate), for example. Medications for seizures and/or pain may be employed, in particular embodiments of the invention. In specific embodiments, a combination of lapatinib and capecitabine is employed, for example.

### III. Detection and Quantitation of RNA Species

One embodiment of the instant invention comprises a method for identification of BCBM cells (in particular EpCAM negative circulating tumor cells (CTCs)) in a biological sample at least in part by amplifying and detecting particular nucleic acids corresponding to at least part of the BCBM gene signature reported herein. The biological sample may be any tissue or fluid in which BCBM cancer cells might be present, but in particular of CTCs. Various embodiments include blood, serum, plasma, lymph fluid, ascites, serous fluid, pleural effusion, sputum, cerebrospinal fluid, lacrimal fluid, stool, breast milk, nipple aspirate, urine, and so forth.

Nucleic acid used as a template for amplification is isolated from cells contained in the biological sample, according to standard methodologies. (Sambrook et al., 1989) The nucleic acid may be genomic DNA or fractionated or whole cell RNA or mRNA. Where RNA is used, it may be desired to convert the RNA to a complementary cDNA. In one embodiment, the RNA is whole cell RNA and is used directly as the template for amplification.

Pairs of primers that selectively hybridize to nucleic acids corresponding to at least part of the gene signature are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Affymax technology; Bellus, 1994).

Following detection, one may compare the results seen in a given patient with a statistically significant reference group of normal patients and prostate, cancer patients. In this way, it is possible to correlate the amount of nucleic acid detected with various clinical states.

A. Primers

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred. Primers may be utilized that respectively target any one of the genes of the signature. Generation of primers is well known in the art, but examples of primers are included in Example 11.

B. Template Dependent Amplification Methods

A number of template dependent processes are available to amplify the nucleic acid sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety.

Briefly, in PCR, two primer sequences are prepared which are complementary to regions on opposite complementary strands of the target nucleic acid sequence. An excess of deoxynucleoside triphosphates are added to a reaction mixture along with a DNA polymerase, e.g., Taq polymerase. If the target nucleic acid sequence is present in a sample, the primers will bind to the target nucleic acid and the polymerase will cause the primers to be extended along the target nucleic acid sequence by adding on nucleotides. By raising and lowering the temperature of the reaction mixture, the extended primers will dissociate from the target nucleic acid to form reaction products, excess primers will bind to the target nucleic acid and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DINA polymerases. These methods are described in WO 90/07641 filed Dec. 21, 1990. Polymerase chain reaction methodologies are well known in the art.

Another method for amplification is the ligase chain reaction (“LCR”), disclosed in European Application No. 320 308, incorporated herein by reference in its entirely. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[.alpha.-thio]-triphosphates in one strand of a restriction site also may be useful in the amplification of nucleic acids in the present invention. Walker et al. (1992), incorporated herein by reference in its entirety.

Strand Displacement Amplification (SDA) is another method of carrying out isothermal amplification of nucleic acids which involves multiple rounds of strand displacement and synthesis, ie., nick translation. A similar method, called Repair Chain Reaction (RCR), involves annealing several probes throughout a region targeted for amplification, followed by a repair reaction in which only two of the four bases are present. The other two bases may be added as biotinylated derivatives for easy detection. A similar approach is used in SDA. Target specific sequences also may be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Still other amplification methods described in GB Application No. 2 202 328, and in PCT Application No. PCT/US89/01025, each of which is incorporated herein by reference in its entirety, may be used in accordance with the present invention. In the former application, “modified” primers are used in a PCR™ like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR. Kwoh et al. (1989); Gingeras et al., PCT Application WO 88/10315, incorporated herein by reference in their entirety. In NASBA, the nucleic acids may be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Davey et al., European Application No. 329 822 (incorporated herein by reference in its entirely) disclose a nucleic acid amplification process involving cyclically synthesizing single-stranded RNA (“ssRNA”), ssDNA, and double-stranded DNA. (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA. These copies may then re-enter the cycle leading to very swift amplification. With proper choice of enzymes, this amplification may be done isothermally without addition of enzymes at each cycle. Because of the cyclical nature of this process, the starting sequence may be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, ie., new templates are not produced from the resultant RNA transcripts. Other amplification methods include “race” and “one-sided PCR™” Frohman (1990) and Ohara et al. (1989), each herein incorporated by reference in their entirety.

Methods based on ligation of two (or more) oligonucleotides in the presence of nucleic acid having the sequence of the resulting “di-oligonucleotide”, thereby amplifying the di-oligonucleotide, also may be used in the amplification step of the present invention Wu et al. (1989), incorporated herein by reference in its entirety.

C. Separation Methods

Following amplification, it may be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

D. Identification Methods

Amplification products must be visualized in order to confirm amplification of the target nucleic acid sequences. One typical visualization method involves staining of a gel with ethidium bromide and visualization under UV light. Alternatively, if the amplification products are integrally labeled with radio- or fluorometrically-labeled nucleotides, the amplification products may then be exposed to x-ray film or visualized under the appropriate stimulating spectra, following separation.

In one embodiment, visualization is achieved indirectly. Following separation of amplification products, a labeled, nucleic acid probe is brought into contact with the amplified target nucleic acid sequence. The probe preferably is conjugated to a chromophore but may be radiolabeled. In another embodiment, the probe is conjugated to a binding partner, such as an antibody or biotin, where the other member of the binding pair carries a detectable moiety.

In one embodiment, detection is by Southern blotting and hybridization with a labeled probe. The techniques involved in Southern blotting are well known to those of skill in the art and may be found in many standard books on molecular protocols. See Sambrook et al., 1989. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

### IV. Kit Components

All or some of the essential materials and reagents required for detecting nucleic acids of the signature in a biological sample may be assembled together in a kit. The kit may comprise preselected primer pairs for nucleic acids corresponding to at least some embodiments of the gene signature. Also included may be enzymes suitable for amplifying nucleic acids including various polymerases (RT, Taq, etc.), deoxynucleotides and buffers to provide the necessary reaction mixture for amplification. Preferred kits also may comprise primers for the detection of a control, non-differentially expressed RNA such as beta-actin, for example.

The kits generally may comprise, in suitable means, distinct containers for each individual reagent and enzyme as well as for each primer pair. Preferred pairs of primers for amplifying nucleic acids are selected to amplify the sequences designated herein as being part of the signature.

In certain embodiments, kits will comprise hybridization probes designed to hybridize to a sequence or a complement of a sequence designated herein as being part of the signature. Such kits generally will comprise, in suitable means for close confinement, distinct containers for each individual reagent and enzyme as well as for each hybridization probe.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A CTC Biomarker Assay to Combat Breast Cancer Brain Metastasis

The identification and characterization of circulating tumor cells (CTCs) inductive of fatal metastasis remains elusive. Because care is essentially palliative once metastasis occurs and drug combinations are rarely tested to reduce established metastasis, new approaches to predict metastatic onset for the development of effective treatments are critical. They can be more profound when coupled with a definition of CTC-associated characteristics. Specifically, the incidence of brain metastatic breast cancer (BMBC) is alarmingly increasing; and BMBC is common in patients negative for estrogen/progesterone receptors and over-expressing epidermal growth factor receptor1 or 2 (EGFR or HER2/neu). However, both traditional and recent therapies using EGFR/HER2 target designs had underwhelming success in the clinical management of BMBC.

The inventors have made four key discoveries shedding new light on the biology of CTCs, and identified useful biomarkers for the development of an assay to predict and guide treatment of BMBC in the clinic. First, the inventors found that CTCs recovered from clinical BMBC specimens rarely express epithelial cell adhesion molecule (EpCAM) and could not be detected by CellSearch™ (Veridex, LLC), a FDA-cleared prognostic CTC test which evaluates only CTCs which are positive for EpCAM. Second, they isolated subsets of CTCs from patients with BMBC by combining technologies alternative to CellSearch™, such as FICTION (BioView™) along with flow cytometry, and a highly sensitive RT-PCR employing a thermodynamically-matched primer design to identify wild type and variant gene expression. They were able to visualize, isolate, and study CTCs that CellSearch™ would never capture. These EpCAM-negative CTCs express a multitude of tumor cell traits, including markers of stemness. Third, they established procedures retrieving viable CTC subsets via fluorescence-activated cell sorting which were amenable to growth in vitro and subsequent analyses. Fourth, they defined EpCAM-negative CTCs to possess the known markers HER2/neu, EGFR, uPAR, ALDH1, cytokeratins, and CD44high/CD24low. However, and of note, there were two additional markers that are highly expressed on EpCAM-negative CTCs isolated from BMBC specimens: Heparanase and Notch1; with evidence that CTCs with this profile are tumorigenic in animals. The above set of biomarkers may be referred to herein as “the BMBC CTC signature,” although in some embodiments a subset of these genes is employed for the signature.

Based on the discoveries, the inventors consider that the BMBC CTC signature, additive to CellSearch™, is of clinical utility for efficacy of treatment by predicting all cases of BMBC; and that heparanase and Notch1 are novel therapeutic targets for personalized patient care. To further characterize this embodiment, the inventors consider the following points that can provide preclinical validation and further characterize the embodiments by identifying heparanase and Notch1 pathways as critical CTC biomarkers for clinical use.

One embodiment is to characterize the CTC signature, and variations of this signature, from patient populations with or without BMBC. One can detect, isolate, and characterize CTCs positive for heparanase and Notch1, and related subsets, from peripheral blood of patients clinically diagnosed with or without BMBC. One can establish CTC lines in culture and investigate them phenotypically.

One embodiment assists in determination of lead CTC biomarker signatures causal of BMBC onset through xenotransplantation studies using immunocompromised mice. One can inject CTC subsets either into the mammary fat pad, the heart, or the carotid artery in order to recapitulate the sequential steps of the metastatic cascade leading to BMBC. One can monitor the development of brain metastasis by magnetic resonance imaging and biomarker investigation.

One embodiment is to characterize the therapeutic potency of CTC-associated heparanase and Notch1 pathways in BMBC by pINDUCER lentivirus. One can characterize of heparanase and Notch1 as targets for clinical intervention. One can employ pINDUCER, a novel inducible shRNA/cDNA expression lentiviral system, and perform heparanase and Notch1 gain-/loss-of-function CTC investigations either in vitro (CTC lines) or in vivo (CTC xenograft models) to characterize their roles in the regulation of BMBC onset.

### BACKGROUND

Brain metastatic breast cancer (BMBC) represents the most devastating and feared consequence of breast cancer since patients with BMBC have an exceptionally poor prognosis. Despite increasing incidence and being recognized as a problem of urgent clinical priority, mechanisms causing BMBC are understudied and remain largely unknown. Human epidermal growth factor receptor1 and 2 (EGFR and HER2/neu, respectively) are predictive of an increased risk for BMBC (EGFR positivity; HER2/neu overexpression) (Lu et al., 2009). An important aspect to combat BMBC is the discovery of circulating tumor cells (CTCs) targeting the brain and their properties. CTCs represent the “seeds” of intractable metastatic cancer, and provide a unique alternative to invasive biopsies for the detection, diagnosis, and monitoring of solid tumors (Cristofanilli et al., 2004; Pantel et al., 2008). However, the characterization of CTC subtypes, CTC heterogeneity and molecular profiling remain elusive. For example, the only diagnostic CTC platform currently approved by the Food and Drug Administration—CellSearch™ (Veridex)—detects only CTCs which are positive for the epithelial cell adhesion molecule (EpCAM) and cytokeratins (CKs), both tumor cell markers (Cristofanilli et al., 2004; Pantel et al., 2008; Hayes et al., 2006), but is unable to capture any other CTC subtypes (e.g., ones from breast cancers with highly aggressive features) (Sieuwerts et al., 2009), investigate properties of viable CTCs, or assay for biomarkers that permit CTC colonization to specific organ sites such the brain.

Several patient and experimental studies indicate that heparanase (HPSE) is a potent pro-tumorigenic, pro-angiogenic, and pro-metastatic molecule, initiating multiple effects which drastically alter the metastatic outcome (Ritchie et al., 2011; Marchetti and Nicolson, 2011; Marchetti and Chen, 2000; Zhang L., Sullivan P. S., Gunaratne P., et al., 2011; Ridgway et al., 2010; Vreys and David, 2007). Heparanase is the only endoglycosidase in mammals cleaving heparan sulfate (HS)—the main polysaccharide of the cell surface and extracellular matrix—into fragments which retain biological activity. An established role of heparanase is to release HS-bound growth and angiogenic factors stored in the extracellular matrix, and to regulate their levels and overall potency. Highest levels of HPSE activity have been consistently detected in cells metastatic to brain, regardless of the cancer type or model system studied (Marchetti and Nicolson, 2011; Marchetti and Chen, 2000; Zhang L., Sullivan P. S., Gunaratne P., et al., 2011). Of relevance, recent findings have demonstrated that heparanase has functions which are independent of its enzymatic activity and mediated by the latent, unprocessed form of the molecule, e.g., promoting cell adhesion, augmenting EGFR phosphorylation, and altering cell signaling (Ridgway et al., 2010; Ridway et al., 2011; Cohen-Kaplan et al., 2008;). The therapeutic disruption of heparanase therefore provides an opportunity to block multiple pathways that control tumor-host interactions and are crucial for tumor cell adhesion, growth, and metastasis.

Heparin has long been known to be an inhibitor of HPSE but its use is limited due to the risk of inducing adverse bleeding complications. However, it has been possible to separate the anticoagulant and antiheparanase properties of heparin through a series of chemical modifications. A modified non-anticoagulant heparinoid that is 100% N-acetylated and 25% glycol split, SST0001, was recently isolated (Ritchie et al., 2011; Casu et al., 2008; Naggi et al., 2005). SST0001 is a small, cell membrane-permeable molecule, and a potent inhibitor of heparanase. Furthermore, its glycol-splitting causes heparin to lose its affinity for antithrombin with a resulting loss of anticoagulant activity (Ritchie et al., 2011). Thus, SST0001 is endowed with unique characteristics to make its use suitable as a cancer therapeutic and is available to us.

Notch signaling is known to be activated in human breast cancer, with the accumulation of Notch1 intracellular domain in tissues (Stylianou et al., 2006). Elevated Notch ligands have been shown to correlate with poor overall survival in breast cancer patients (Dickson et al., 2007). Notch signaling plays a role in stem cell maintenance (Dontu et al., 2004; Bouras et al., 2008), and may contribute to the maintenance of the cancer stein cell phenotype, with the strongest evidence in breast cancer (Bolos et al., 2009; Pannuti et al., 2010; Kakarala and Wicha, 2007; Farnie and Clarke, 2003). Of note, two recent studies have asserted Notch1 relevance in BMBC. In the first study (Nam et al. 2008). MDA-MB-435 carcinoma cells, selected for metastatic growth in the brain, exhibited an upregulation of the Notch1 pathway compared to parental counterparts, and that either the commercial γ-secretase inhibitor DAPT or the RNA interference-mediated knockdown of Notch1 inhibited tumor cell migration and invasion in vitro. In the second study (McGowan et al., 2011), an experimental in vivo BMBC model was used to assess the role of the Notch1 pathway. Using two different experimental strategies, Notch signaling inhibition significantly prevented the colonization of brain metastatic MDA-MB-231 human breast cancer cells in the brain. These authors also determined the relationship of the “stem-like” phenotype (CD44hi/CD24lo) to both brain metastasis and Notch1 signaling inhibition, nominating Notch1 as a potential therapeutic target for inhibition of breast cancer brain metastasis (McGowan et al., 2011).

### Embodiments of the Invention

The present invention provides functional profiling of heparanase, Notch1, and correlative biomarkers, in CTC subtypes detected, isolated, and characterized from blood of BMBC patients. The scope is to develop a CTC biomarker assay useful to predict BMBC and/or prevent further metastases (relapse free survival). Results expand the development of this CTC-based assay to predict and/or provide new drug combinations to treat BMBC. In specific embodiments, there is a novel concept for an EpCAM-negative but HPSE/Notch1 positive CTC signature (“The BMBC signature”) useful to predict and monitor BMBC for personalized patient care.

Initially, the inventors selected patients either possessing or not possessing BMBC (clinical diagnosis) for CTC investigations. Peripheral blood was retrieved from these two patient populations which underwent CTC analyses using the CellSearch™ system from Veridex (a Johnson & Johnson Company). By employing this system, the inventors observed that CTCs, defined as EpCAM+/CKs+ cells which are negative for CD45, a hematolymphoid marker (Cristofanilli et al., 2004; Pantel et al., 2008; Hayes et al., 2006; Sieuwerts et al., 2009), were largely undetectable in almost 65% of patients possessing BMBC and having HER2 amplified disease or being triple negative (estrogen receptor/pro-gesterone receptor/HER2 negative)(Bos et al., 2009; Hicks et al., 2006; Smid et al., 2008); while the opposite was found in patients without BMBC disease, as diagnostically assessed (Table 1).

TABLE 1
CTC detection in BMBC vs. non-BMBC patients using CellSearch ™

| | | Cell Search ™-Veridex | | | | | |
|---|---|---|---|---|---|---|---|
| | | CTC = 0 | | CTC = 1 | | CTC > 1 | |
| | n | % | | % | | % | |
| BMBC | 17 | 64.7* | (11/17) | 0.06 | (1/17) | 29.4 | (5/17) |
| No BMBC | 14 | 35.7 | (5/14) | 14.3 | (2/14) | 50.0 | (7/14) |

*p < 0.01

Next, the inventors aimed to interrogate a wider spectrum of CTCs, extending the definition of EpCAM positivity which represent the primary selection step (EpCAM-coupled iron particles followed by magnetic separation) for the identification of CTCs by CellSearch™ (Cristofanilli et al., 2004; Pantel et al., 2008; Hayes et al., 2006; Sieuwerts et al., 2009). To this end, they employed a technology, termed FICTION, that consists of performing immunofluorescence (IF) analyses for specific membranous, cytoplasmic, or nuclear antigenic markers, coupled with fluorescence in situ hybridization (FISH) to detect gene amplification on the same cells; FICTION is then combined with quantification of signals via an automated scanning instrument (the Duet-3™ system; BioView, Ltd.). The BioView™ system is capable of scanning thousands of cells, visualizing and classifying rare cancer-associated circulating cells according to specific IF/FISH patterns on a per-cell basis. These approaches have been recently validated (Katz et al., 2010), allowing one to investigate a much larger number of CTCs (several orders of magnitude higher than ones recognized by CellSearch™), and to categorize CTCs being of epithelial, mesenchymal, or stem-cell in origin (Katz et al., 2010; Polyak and Weinberg, 2009); Khanna et al., 2010; Marrotta and Polyak, 2009). An additional advantage of BioView™ is the ability to investigate CTC biomarkers (by IF), along with aneuploidy or amplification (by FISH) for specific genes, e.g., EGFR and HER2/neu (Bos et al., 2009; Hicks et al., 2006), in the same cells.

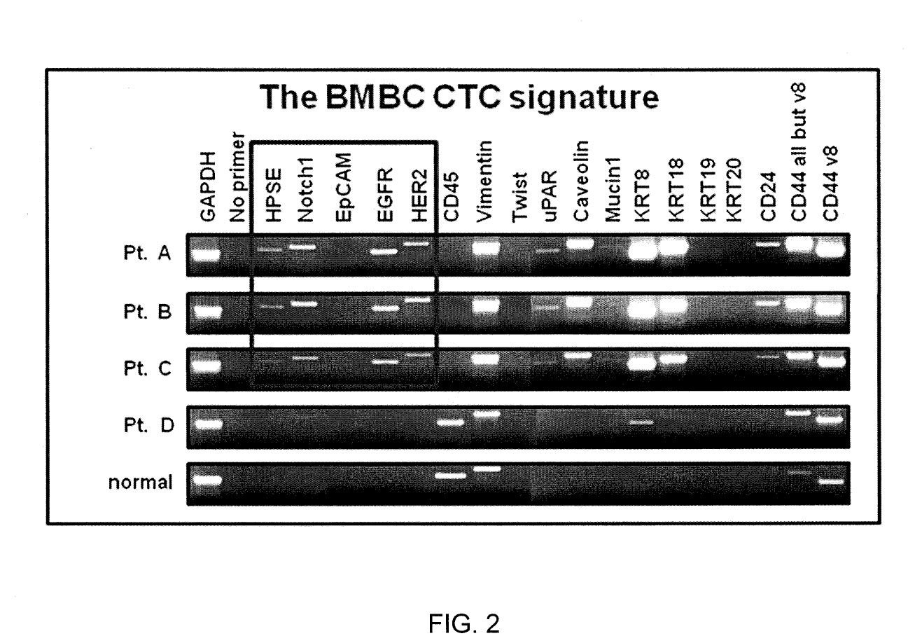

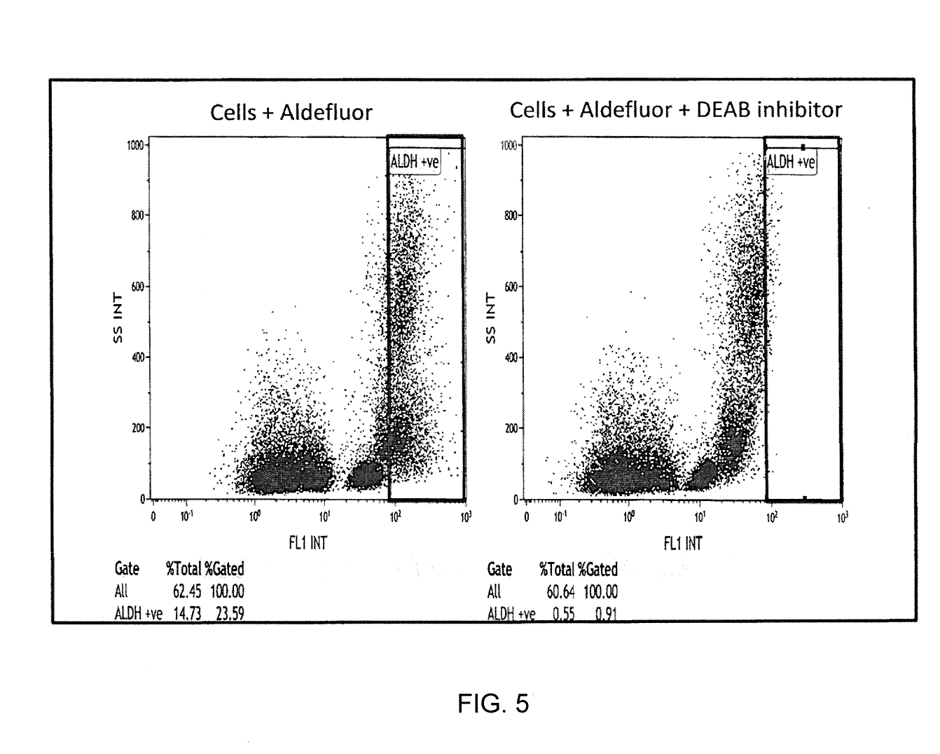

The inventors have established the feasibility of FICTION in brain metastatic breast cancer by performing CTC analyses on peripheral blood mononuclear cells (PBMCs) isolated from blood of BMBC patients. The inventors consistently observed high levels of EGFR amplification (FIG. 2) independent of HER2/neu status (Bos et al., 2009; Hicks et al., 2006; Smid et al., 2008). Second, by using the Bioview™ scanner system, the inventors were able to visualize and quantify EpCAM-positive CTCs from patients with BMBC as well as human breast cancer cells (SK-BR-3 line) which were spiked in blood of healthy donors (control). For example, of 5,184 PBMCs deposited on a slide from one BMBC clinical case and subsequently scanned, the percentage of PBMCs expressing EpCAM, but negative for the hematolymphoid marker CD45 (Cristofanilli et al., 2004; Hayes et al., 2006; Sieuwerts et al., 2009), was 11.22% or 56,000 EpCAM-positive cells/ml of blood. The number of EpCAM-positive CTCs detected by BioView™ was three orders of magnitude higher than one (21 CTCs/ml of blood) obtained from CellSearch™ CTC analyses using the same specimen. Third, the presence of CTCs positive for CKs but negative for EpCAM and CD45 was also detected. These findings were confirmed by CTC testing of blood from four BMBC patients. Fourth, by applying FICTION on PBMCs isolated from the blood of healthy donors, they were negative for EGFR and HER2/neu aneuploidy, and for the expression of CKs, HPSE and the known stem cell marker aldehyde dehydrogenase1 (ALDH1 (Ginester et al., 2007; Jiang et al., 2009; Khanna et al., 2010) proteins (FIG. 5, top panel) and related transcripts. Conversely, the inventors detected presence of HPSE in CTCs from BMBC patients in conjunction with the expression of ALDH1. The intranuclear localization of HPSE in CTCs, possibly reflecting nucleolar HPSE (Zhang L, Sullivan P, Suyama et al., 2010), was also observed, which correlated with high EGFR amplification within nuclei of the same cells.

The inventors applied BioView™ technologies and discovered a significant correlation between the expression of ALDH1, HPSE, and elevated EGFR amplification in CTCs from BMBC patients: a major proportion of CTCs possessing EGFR amplification was also positive for HPSE (76%) and ALDH1 (65%) (Table 2).

TABLE 2
Characterization of CTCs in BMBC

| | % CTCs with EGFR gene amplification |
|---|---|
| Pattern of Antigen Expression | (mean of total analyzed) |
| HPSE+/ALDH+ | 47.1** |
| HPSE+/ALDH⁻ | 29.3** |
| HPSE−/ALDH+ | 18.3 |
| HPSE−/ALDH− | 5.3 |

**= p < 0.01

Again, the vast majority of CTC subtypes quantified by the Bioview™ system could not be captured by a parallel CTC testing using the CellSearch™ platform which detects only a very small proportion of CTCs (Sieuwerts et al., 2009), and only CTCs which are positive for EpCAM and CKs (Cristofanilli et al., 2004; Hayes et al., 2006; Sieuwerts et al., 2009). Therefore, the BioView™ platform not only captures more EpCAM-positive CTCs than CellSearch™ but also CTC subtypes—different from one CellSearch™ is able to identify which possess varying levels of EpCAM, e.g., EpCAM-negative CTCs. However, it must be noted that the inventors discovered an extensive CTC heterogeneity (see also Marusyk and Polyak, 2010) which precluded them from achieving significant correlation values, e.g., between levels of EpCAM and CKs, and expression of HPSE and ALDH1 in CTC populations. This indicated that the selection of defined CTC subsets, and their characterization, is highly relevant.

For this purpose, and to interrogate CTC biomarker expression further, the inventors sorted CTCs for EpCAM, ALDH1, and HPSE, and established procedures for retrieving viable CTC subsets amenable to growth in vitro and subsequent analyses (CTC lines). The inventors used FACS StarPLUS, a flow cytometric instrument which allows the simultaneous quantitative analysis of up to 12 parameters (12 fluorescence channels), each of which is assayed at the individual cell level, facilitating high content analysis of mixed cell populations and rare cell types. The information derived from such multiparameter approach was then complemented by the ability to isolate the desired cell population with high-speed cell sorting for culture and further characterization using cellular and molecular biology techniques. Peripheral blood mononuclear cells (PBMCs) were isolated by Ficoll-Hypaque centrifugation using PB samples (30-40 mls) drawn from patients possessing BMBC. PBMCs were subsequently analyzed by flow cytometry/FACS. Next, by developing a highly sensitive RT-PCR using a thermodynamically-matched primer design for parallel identification of multiple wild type and variant gene expression, they were able to detect markers in sorted CTC subsets that CellSearch™ would never capture. These EpCAM-negative CTCs expressed a multitude of tumor cell traits, including additional markers of stemness and neoplasticity. For example, the inventors found EpCAM-negative CTCs to possess urokinase plasminogen activator receptor (uPAR), vimentin, cytokeratins (KRTs), and CD44^{high}/CD24^{low} (Sieuwerts et al., 2009). Of note, besides EGFR and HER2/neu, two markers stood-out: Heparanase (Marchetti and Nicolson, 2001; Marchetti and Shen, 2000; Zhang L., Sullivan P. S., Gunaratne, 2011) and Notch1 (Park et al. 2010; McGowan et al. 2011); with evidence that CTCs with this profile are tumorigenic in animals and recapitulate the clinical presentation of human BMBC disease. The above set of biomarkers has been termed “The BCBM CTC signature”. Further, they were able to culture in vitro CTC subsets from BMBC patients following FACS selection, and establish respective CTC lines. These CTCs were further sorted for EpCAM and Notch1 overexpression (a low detection of cell membrane HPSE precludes its selection by FACS). EpCAM-negative Notch1 overexpressors were recovered and grown in tissue culture. These overexpressors were then again FACS-sorted (EGFR and HER2/neu) to obtain EGFR/HER2 CTC overexpressors. EpCAM-negative, Notch1/EGFR/HER2 CTC overexpressors retrieved from FACS were viable, could be grown in tissue culture for subsequent in vitro characterization (e.g. expression of heparanase, cell morphology, adhesive and invasive abilities, etc.), and showed metastatic competency in vivo, since able to form BMBC once injected in severely immunocompromised animals.

Research Strategy Embodiments

A confluence of data from studies of human cancer shows that characterizing properties of CTCs is of paramount importance, and considered a fundamental advancement to combat metastatic death and improve understandings of the biology of cancer metastasis. Although the precise definition of distinct CTC subtypes was elusive, the inventors have discovered clues implicating biomarkers, e.g., heparanase and Notch1, in CTCs isolated from blood of patients with BMBC. One can execute the following exemplary research strategies to characterize embodiments of the invention.

Characterize the CTC Signature, and Variations of this Signature, from Patient Populations with or without BMBC.

This can be accomplished as follows: First, one can amplify investigations of BMBC CTC biomarkers by increasing the number of BMBC clinical cases to be investigated for the CTC signature. One can analyze PBMCs isolated from patients' blood using FACS, IF and FISH; however, and of note, one can also employ the new DEPArray platform. The DEPArray™ (Silicon Bio-systems, Inc.) is a cell-based microarray for the individual detection and recovery of viable, rare cells in blood. e.g. CTCs. It utilizes image-based selection of cells from small cell loads to isolate cells of interest at the single-cell level with 100% purity and viability. Unlike conventional methodologies such as FACS, the DEPArray™ technology separates and manipulates cells individually, in sterile conditions, allowing cell culturing and downstream molecular and genetic analyses. The DEPArray is driven by a microelectronic silicon-substrate-embedded control circuitry which addresses each individual dielectrophoretic (DEP) cage in a chip. This results in unprecedented flexibility and selectivity, and represents a breakthrough in biological research. The tiny electrodes (300,000) on the chip surface (20 μm×20 μm) permit DEP cages to accommodate as little as one single cell, enabling the parallel individual manipulation of up to 100,000 cells. These DEP cages enable sterile cell capturing and their routing by a regulation of the electric field. The selection and identification of CTCs, or other rare blood cells, is then accomplished through fluorescence microscopy assessing a multi-parametric image-based selection which allows the recovery of 100% pure cells. An additional important aspect of this system is that cells isolated in this manner maintain their viability. DNA integrity, and proliferation abilities. Therefore, the DEPArray™ isolation of CTCs is not only compatible with upstream CTC enrichment and/or CTC visualization by CellSearch™ but also enables the recovery of viable CTCs to further investigate molecular and genetic signatures at a single-cell level. To confirm BMBC biomarker specificity, one can employ PBMCs from blood of patients with primary breast cancers that have not metastasized to brain (non-brain metastatic controls). One can evaluate blood from 40 patients with BMBC and an equivalent number of cases without BMBC (control group) to achieve statistical validity. Blood samples (30-40 mls) from each patient can be collected at baseline—before starting any systemic therapy. To avoid contamination with epithelial cells from the skin, samples can be obtained at the middle of vein puncture after the first 5-10 mls of blood are discarded. These human specimens may be matched for clinical stage and histologic subtype. Blood specimens undergo PBMCs isolation by Ficoll-Hypaque gradient, PBMCs are analyzed by FACS/BioView™, and CTC subsets are further analyzed by the DEPArray™; with CTCs assayed for the expression of HPSE, ALDH1, Notch1, EpCAM, CKs, and CD45 (last three markers are used to define CTCs detected by the CellSearch™ platform; Cristofanilli et al., 2004; Pantel et al., 2008; Hayes et al., 2008; Sieuwerts et al., 2009). This is accomplished by immunofluorescence (IF) staining, in concurrence with studying EGFR and HER2/neu gene amplification by FISH. Further, one can perform parallel CTC testing by CellSearch™ (control experiments). For example, one can employ patients' peripheral blood (7.5 mls aliquot drawn in CellSave™ tubes) for CTC profiling procedures (CPK kit and method) enriching for EpCAM-positive CTCs. The enriched CTC preparations then undergo isolation by the DEPArray™ system, with subsequent characterization of the biomarkers indicated above. Results from the two groups of patients are compared to derive molecular signatures that can be characteristic of CTCs of breast cancers metastasizing to brain. One can obtain freshly drawn blood samples under an IRB-approved protocol from a cohort of patients with clinical evidence of BMBC, and patients without clinical evidence of BMBC. All clinical information concerning specimens can be available, such as TNM staging, histology, and IHC profiles of their primary breast cancers along with test results of HER2/neu expression, estrogen and progesterone receptors (ER, PR) status, histopathological reports of nuclear grade, Ki-67, and EGFR content. HER2/neu overexpressors, EGFR-positive, and triple-negative (ER⁻, PR⁻, HER2/neu⁻) breast cancer subtypes are known to have an increased risk for brain metastasis (Bos et al., 2009: Hicks et al., 2006; Smid et al., 2008), and considered hallmarks of the BMBC phenotype (Hicks et al., 2006). Priority can be given studying these cases. Clinical data are cross-referenced with ones obtained using the DEPArray™ and CellSearch™ platforms, e.g., clinical and radiographic status of the patient according to whether she is positive or negative for BMBC onset. By antigen-independent, quantitative FISH-based assays can detect genetically abnormal sub-populations of CTCs in cancer patients which can be further analyzed for protein markers. Further, by applying the DEPArray™ procedures, the study design enables investigations of CTCs at a single cell level, and the rigorous screening to define the precise properties of CTCs subsets and metastasis-founder CTCs. DEPArray procedures are able to detect CTC numbers higher—at minimum two to three orders of magnitude—than ones obtained using CellSearch™ (Cristofanilli et al., 2004: Hayes et al., 2008; Sieuwerts et al., 2009), in certain embodiments of the invention. Similar to FICTION/BioView™, one can categorize CTCs being of epithelial, mesenchymal or stem-cell in origin (Polyak and Weinberg, 2009; Ginester et al. 2007; Jiang et al. 2009; Khanna et al. 2010). Statistical analyses to detect the analytical differences between the two groups of patients are performed.

Second, PBMCs from BMBC/non-BMBC cases are investigated in parallel utilizing CellSearch™ to compare numbers of CTCs. [positive for EpCAM and CKs but negative for CD45 presence (Cristofanilli et al., 2004; Pantel et al., 2008; Hayes et al., 2008; Sieuwerts et al. 2009)], with ones obtained applying the DEPArray™ and IF/FISH analyses. Results are analyzed and combined with RT-PCR analyses using thermodynamically-matched primers for a multitude of neoplastic and stem cell markers. They are implemented to confirm the CTC status, to compare data with CellSearch™ immunomagnetic separation (e.g., amplification for CKs and CD45), to assess a potential illegitimate gene transcription, e.g., by normal leukocytes (Aktas et al. 2009; Paterlini-Brechot and Benali, 2007), and to decipher CTC biomarkers. One can thus acquire an accurate definition of CTC biomarker expression in relation to: a) EGFR and HER2/neu amplification by FISH; b) EpCAM profiling to discriminate EpCAM levels and their variation; c) EGFR/HER2/neu, HPSE, ALDH1, and Notch1 protein expression in these samples; d) EGFR and HER2/neu profiling in EpCAM-positive CTCs employing respective CellSearch™ CTC kits (Veridex, LLC).

Determining Lead CTC Biomarker Signatures Causal of BMBC Onset Through Xenotransplantation Studies Using Immunocompromised Mice.

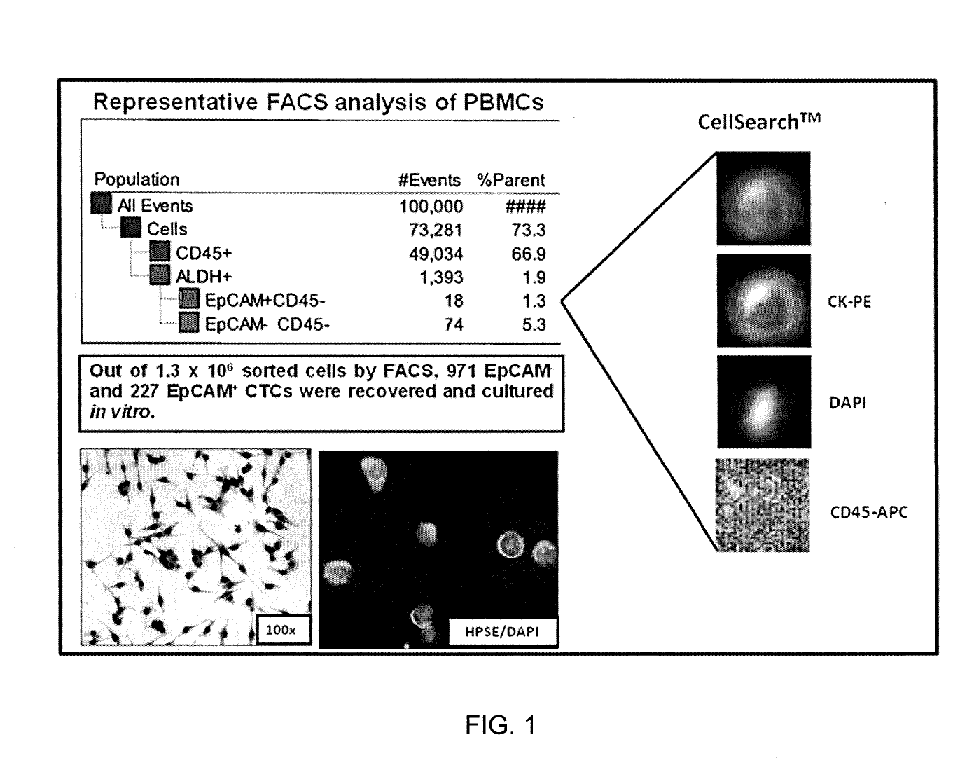

This embodiment can be accomplished as follows: First, one can complement the aforementioned studies by performing fluorescence-associated cell sorting (FACS) analyses of PBMC samples from BMBC to sort CTCs according to HPSE, ALDH1, Notch1, and EpCAM/CKs/CD45 expression. Percentages of CTCs positive for HPSE, ALDH1, Notch1 are determined by gating and enumerating CTC subtypes that display a differential staining for HPSE, ALDH1. Notch1, along with co-expression of CKs but the absence of EpCAM and CD45 (Cristofanilli et al., 2004; Pantel et al., 2008; Hayes et al. 2008; Sieuwerts et al., 2009). One can spike human BMBC cell lines (MB-231BR and MB-231BR3; 9, 15, 26) and/or HER2/neu-positive SK-BR-3 (Sieuwerts et al. 2009) into normal blood from healthy donors (controls). As additional control, one can employ human blood from healthy donors (Gulf Coast Regional Blood Center) to detect baseline levels and the percentage of cells staining positive for CD45, etc., as recovered from flow cytometric/FACS analyses. The inventors have already accrued data supporting the feasibility of these approaches using PBMCs isolated from BMBC patients (triple negative cases). FIG. 1 shows flow cytometric data of PBMCs following their purification via Ficoll-Hypaque gradients, indicating the presence of a subset of CTCs which are positive for ALDH-1; however, negative for EpCAM and CD45. Of note, when the inventors analyzed this FACS-retrieved CTC subset (from the same patient and spiked into normal blood) by CellSearch™ methodology, consisting in the EpCAM-immunomagnetic capture step followed by immunofluorescence for CKs/DAPI positivity but CD45 negativity, the inventors confirmed detection of CTCs (right panels of FIG. 1), of which only two (<1%) were EGFR-positive CTCs. Conversely, flow cytometric analyses of PBMCs from blood of BMBC patients, assessed the presence of a CTC subtype which was negative for EpCAM and CD45, but positive for ALDH1 activity (Aldefluor assay) (NOTE: EpCAM and ALDH1 are not expressed by normal cells) (Ginester et al., 2007: Jiang et al., 2009; Khanna et al., 2010) (Meerbrey et al., 2011) were not detectable by CellSearch™ (FIG. 1 and Table 1). One can perform additional flow cytometric analyses and sort cells according to EpCAM, EGFR, HER2/neu, HPSE, Notch1, and CKs⁺/CD45⁻ expression status. One can use the FACSStarPLUS instrument possessing 12-parameter capabilities (12-fluorescence channels) to assess: a) high HPSE and Notch1 positivity (by immunostaining) and high activities for HPSE (9, 15) and ALDH1, latter by the Aldefluor assay (Ginester et al., 2007; Jiang et al., 2009: see FIG. 5): b) the viability of cells by performing 7AAD viability assays (Sieuwerts et al., 2009); c) percentages of EpCAM⁺/CKs⁺/CD₄₅⁻ cells by CellSearch™ CTC testing (Cristofanilli et al. 2004; Hayes et al., 2008: Sieuwerts et al., 2009) to compare and validate results from the two procedures (FACS-CellSearch™).

Second, one can perform cell adhesion, proliferation, and invasion assays using the isolated CTC subsets grown in vitro as established CTC lines. Notably, to determine CTC invasive values, one can employ a blood-brain barrier transmigration model, consisting upon the use of pure populations of human astrocytes (Clonetics, Inc.) and brain endothelial cells (HBMEC; Ridgway et al., 2011). One can assess the in vitro capabilities of CTC lines to adhere and invade, either alone, or in response to exogenously added latent or active heparanase, forms of the molecule which are known to differentially alter adhesion and invasion events of clonal human BMBC cells (Ridgway et al., 2011: see also Ridgway et al., 2010; Cohen-Kaplan et al., 2008). Third, one can inject CTC subtypes identified from flow cytometry/FACS into severe combined immunocompromised (SCID/Beige) mice. One can inject CTC subsets either into the mammary fat pad, the heart (intracardiac injections), or the internal carotid artery (intracarotid injections) to recapitulate the sequential steps of the metastatic cascade leading to BMBC. Further, one can deliver SST0001 (a potent inhibitor of HPSE activity; Ritchie et al. 2011; Naggi et al., 2005) and/or DAPT (a Notch1 signaling inhibitor; McGowan et al., 2011) in distinct animal subgroups via Alzet pumps delivery. One can monitor the development of brain metastasis according to these treatments by magnetic resonance imaging (MRI) and biomarker identification. MRI investigations may be performed, and one an assess the presence and degree of CTC-induced metastatic onset in these animal subgroups. Once brain metastases are identified, one can euthanize mice and examine brain tissue for BMBC incidence, number of micro- and macrometastases, and expression of above biomarkers (Pantel et al., 2008; Ritchie et al., 2011). One can also inject distinct animal subgroups with highly aggressive human GFP-labelled BMBC cell lines, e.g., MB-231BR-3 (positive control: Zhang L. Sullivan P. S., (Gunaratne et al., 2011; Zhang L, Sullivan P, Suyama et al., 2010), and in the presence or absence of SST0001 treatment (Ritchie et al. 2011; Naggi et al., 2005). One can then monitor BMBC onset in the various animal subgroups by imaging/MRI. Once BMBC are identified, one can euthanize mice and examine brain tissue for BMBC onset, its incidence, and the number/size of brain metastasis. HPSE and EGFR expression levels and their regulation by SST0001 are also determined. Serial section slides are examined by pathologists blinded to the different experimental groups. Three independent experiments may be performed to evaluate: 1) the extent of BMBC in animals injected with HPSE+/ALDH1+CTCs; 2) levels of BMBC and associated markers e.g., HPSE, EGFR, ALDH1, CD44^{high}/CD24^{low}, CD133, etc., in relation to other CTC subtypes injected into animals (e.g., HPSE⁺/Notch1⁻, HPSE⁻/Notch1⁺, etc.); 3) the modulation of BMBC onset and markers expression in animal groups treated with SST0001 and/or DAPT. Statistical analyses are then applied to validate results significance (ANOVA analyses; SAS/STAT 9 User's Guide, 2002).

Define the Therapeutic Potency of CTC—Associated HPSE and Notch1 Pathways in Breast Cancer Brain Metastasis by pINDUCER Lentivirus.

This embodiment may be accomplished as follows: First, one can assess the relevance of heparanase and Notch1 pathways by interrogating the CTC lines that were derived. To this end, one can employ a novel inducible lentiviral system, pINDUCER, which enables tracking of viral transduction and shRNA or cDNA induction of mammalian genes, either in cultured cells or xenografts (Meerbrey et al., 2011). These pINDUCER vehicles achieve a uniform, dose-dependent, and reversible control of gene expression across heterogenous cell populations via fluorescence-based quantification of reverse tet-transactivator expression. Upon the addition of doxycycline (dox), transcription of the turboRFP-shRNA cassette or the cDNA is activated (Meerbrey et al., 2011). This is of relevance because the pINDUCER system can provide a temporal and reversible control of Notch1 and HPSE gene expression in BMBC-associated CTCs, and validation of these biomarkers as regulators of BMBC onset, either independently or in combination. One can study additional blood specimens from patients with or without BMBC, sort CTCs for HPSE and Notch1, and obtain the four HPSE/Notch1 combinatorial CC subsets. One can then grow CTC subsets in vitro and perform sorting for EGFR and HER2 to select CTC subsets containing these additional biomarkers. One can confirm the gene/protein expression by RT-PCR and IF/Western blotting, respectively.

Second, one can test the effectiveness of the pINDUCER lentivirus toolkit for inducing loss-of-function of HPSE and Notch1 in HPSE+/Notch1+ CTCs. Detailed maps of pINDUCER constructs are available (Meerbrey et al., 2011). Biological endpoints are the regulation of HPSE/Notch1 expression using the pINDUCER system in CTC subsets in vitro, and the modulation of BMBC following the injection of pINDUCER-transduced CTC subsets in animals. Specifically, one can use pINDUCER11 (miR-RUG) lentivirus, encoding a constitutive cassette (rtTA3 and eGFP) and shRNAs targeting either HPSE or Notch1, in addition to using scrambled control vectors (Meerbrey et al. 2011). ShRNAs are cloned in Xho/Ml1 from GIPZ clones. One can transduce HPSE+/Notch1+CTC subsets with vectors (m.o.i.=3), and analyze them for cellular fluorescence by FACS, and for transcript expression of Notch1 and/or HPSE, respectively. Lastly, one can inject transduced CTCs into immunocompromised SCID/Beige mice via mammary pad, intracardiac, or intracarotid injection routes. Controls may comprise performing the same experiments however employing untransduced CTC subsets and/or CTCs transduced with scrambled vector controls. Animals are then administered doxycycline (dox+) or vehicle (dox−), and monitored for BMBC onset. At set time point, animals are sacrificed and brains analyzed for the presence of BMBC. Serial sectioning is performed and the presence and number of brain micro- and macro-metastasis is then determined (Gril et al., 2008).

Third, one can apply the above approaches yet investigate the gain-of-function of HPSE and Notch1 in HPSE-Notch1-CTCs. To this end, one can employ the pINDUCER20 (ORF-UN) lentivirus to induce HPSE and/or Notch1 cDNAs (Gril et al., 2008). CDNA cloning may be accomplished using standard Gateway recombination. HPSE-/Notch1-CTC subsets are transduced with pINDUCER20-eGFP and pINDUCER20-HPSE (or Notch1), selected for neomicin resistance, cultured with or without dox, and analyzed by flow cytometry and Western blotting to reveal the presence of HPSE and/or Notch1 expression. Secondly, one can inject transduced CTCs into SCID/Beige mice, animals are administered dox or vehicle (no dox), and BMBC onset are monitored and assessed per above (see also Zhang L, Sullivan P, Suyama et al., 2010). Data of all experiments is subsequently analyzed for statistical significance using ANOVA with experiment specified as the random effect (SAS/STAT 9 User's Guide, 2002).

In embodiments of the invention, there is a correlation between HPSE and Notch1 presence. One can also confirm the elevated expression of heparanase and ALDH1, coupled with high EGFR amplification, in those CTCs isolated from clinical cases of BMBC, and to demonstrate a significant correlation between HPSE and ALDH1 expression in BMBC vs. non-BMBC cases, in certain embodiments of the invention. Further, one can relate findings of high heparanase expression in CTCs from breast cancer cases being high HER2/neu expressors (Lu et al. 2009; hicks et al., 2006), negative for ER, PR, and HER2/neu (triple negatives) (Sieuwerts et al., 2009), or EGFR-positive (Rimawi et al., 2010), in specific embodiments. These subtypes are known to possess increased propensities to colonize the brain (Lu et al., 2009; Hicks et al. 2006). In particular embodiments, FACS-sorted CTC subtypes colonize the brain (e.g., highest for HPSE⁺/ALDH1⁻/Notch1⁺ and EGFR/HER2/neu) once injected into animals, compared to related controls, e.g., animals injected with the HPSE⁻/ALDH1⁻/Notch1⁻ CTCs subgroup, and combinatorial. Overall, these results yield an accurate portrait of molecular signatures of BMBC that can greatly aid breast cancer patient prognosis and treatment prior to brain metastatic onset. Secondly, one can expect to identify Notch1 and HPSE as important biomarkers for the development and progression of BMBC. The inventors anticipate that the pINDUCER system robustly suppresses Notch1 and HPSE expression in vitro and BMBC in vivo following Notch1/HPSE shRNA insertion and induction, while the opposite is expected to occur using vectors with cDNA induction for Notch1 and/or HPSE. In at least some cases, the most striking effects in the BMBC phenotype are observed when both markers are present. One can evaluate contributions of HPSE/Notch1 pathways in BMBC onset when only one marker is knocked-down or induced.

CellSearch™ CTC Testing.

Patient peripheral blood samples (PB: 7.5 mls) collected in CellSave™ tubes are analyzed (CellSearch™, Veridex). PB is diluted with buffer, and samples loaded onto the CellTracks AutoPrep™ system. CellTracks aspirates plasma, and adds anti-Epithelial Cell Adhesion Molecule (EpCAM) ferrofluid to enhance magnetic incubation of cells. Second, the system aspirates unmagnetized cells, and then stains cells with anti-CK-PE to identify intracellular cytokeratin-8, -18 and/or -19, anti-CD45/APC to identify leukocytes, and DAPI to stain cell nuclei. Finally, samples are loaded onto CellTracks™ cartridges for analysis by the CellTracks AnalyzerII™. This instrument scans CellTracks cartridges for CTC events and present them for CTC visualization, determination, and enumeration. A CTC is defined by this procedure as an intact, morphologically round cell with a defined nucleus/cytoplasm ratio (approximately 0.8) that stains positive for DAPI and CK-PE, respectively but is negative for CD45/APC, a marker for leukocytes (Cristofanilli et al., 2004; Hayes et al. 2008; Sieuwerts et al., 2009). CTC enumeration is determined for each individual sample and may be provided as CTC counts, per the above definition.

Statistical Analyses.

One can use the Spearman χ² test for distributional differences between BMBC patients and controls according to categorical variables, and the Mann-Whitney test to determine differences in continuous variables (SAS/STAT 9 User's Guide, 2002). The Mann-Whitney test may also be used to test for differences in each CTC biomarker between BMBC and non-BMBC patients and controls. Simple linear regression analyses are performed to test for trends in the biomarkers by disease. P values are used to determine the level of significance for each test. A one-sided p value of <0.05 may be considered statistically significant (SAS/STAT 9 User's Guide, 2002).

Statistical Considerations.

1) Endpoints: Experimental endpoints can generally be set at 4-6 weeks after the intracardiac injection of CTC subtypes. Based upon experience, brain metastasis in control animals e.g., injected with human BMBC cell lines (Zhang L, Sullivan P, Suyama, 2010), at this time are of adequate size for comparison of growth characteristics with the experimental groups which the inventors consider will have a significantly different number and size of brain metastasis than controls. 2) Randomization: Study animals are controlled by age, weight and any possible experimental condition, e.g., time of cell line injected, room temperature, food and water. On the day of tumor cell injections, experimental animals are randomly assigned to either a control group or to a study group. The biostatistician may generate a randomization list, and the randomization ratio is 1:1 with an equal number of animals in each group. 3) Sample size and study power, one can use several study groups using animals: one group can serve as control, e.g., animals with no SST0001 administration. Sample size and study power are determined by the two-sided Fisher's exact test with 5% type I error (SAS/STAT 9 User's Guide, 2002). 4) Data analyses: Analysis of variance (ANOVA) is performed for these in vivo experiments (SAS/STAT 9 User's Guide, 2002). It can be used to examine the primary endpoint of tumor size at time of sacrifice. Tumor size can be summarized for each group using mean, standard deviation, median, and range. Secondly, data are analyzed using appropriate statistical formulas and software to achieve a complete understanding of results significance. P values less than 0.05 may be considered statistically significant. BMBC slides are analyzed and IHC staining scores for heparanase and/or other BMBC markers are provided. Staining scores can range from 0 to 4+ as follows: negative (0), weakly positive (1+), moderately positive (2+), positive (3+), and strongly positive (4+). Any slides staining ≧1+ may be considered positive. Results are tabulated and statistically analyzed for all patient samples.

### Example 2

### The Identification and Characterization of Breast Cancer CTCs Competent for Brain Metastasis

Brain metastatic breast cancer (BMBC) represents the most feared consequence of breast cancer because uniformly fatal and increasing in frequency at alarming levels. Despite its devastating outcome, mechanisms causing BMBC remain largely unknown. Similarly, properties and biomarker identification of circulating tumor cells (CTCs), the “seeds” of metastatic disease remain elusive. Here the inventors report novel strategies investigating CTCs isolated from peripheral blood mononuclear cells (PBMCs) of patients with BMBC, including the development and characterization of CTC lines. The inventors identified a unique CTC signature (HER2+/EGFR+/HPSE+/Notch1+/EpCAM−) investigating CTCs that could not be captured by the FDA-approved Veridex CellSearch™ platform (EpCAM-negative CTCs). Second, the inventors analyzed the invasive and metastatic competencies of isolated CTCs. Established CTC lines over-expressing the signature were highly invasive and capable to generate brain metastasis in xenografts. Third, tumor cell morphologies of CTC-induced metastases closely resembled those of pathologically assessed tumors of patients whose blood was source for CTC isolation. Fourth, the expression of proteins of the CTC signature was detected in CTC-induced BMBC. Collectively, the inventors provide first-time evidence of human CTCs isolation using the signature and long-term growth by establishing CTC lines, and CTC metastatic competency in the presence of a marker signature necessary to promote BMBC.

### Example 3

### Detection of Circulating Cells with EGFR Gene Amplification and Expression of HPSE and ALDH1

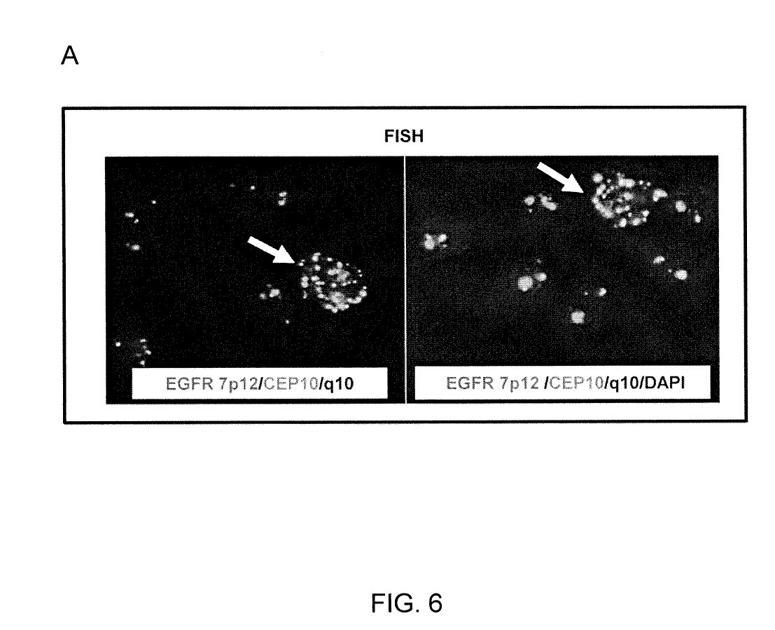

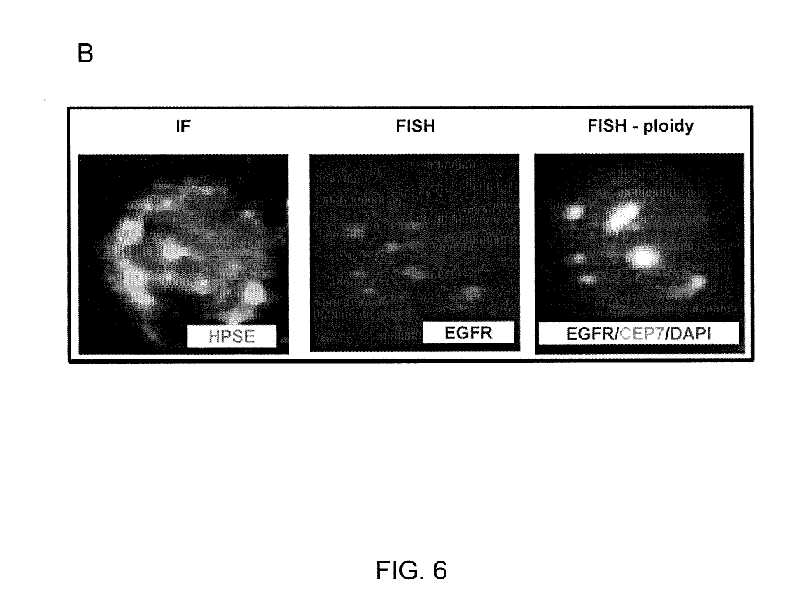

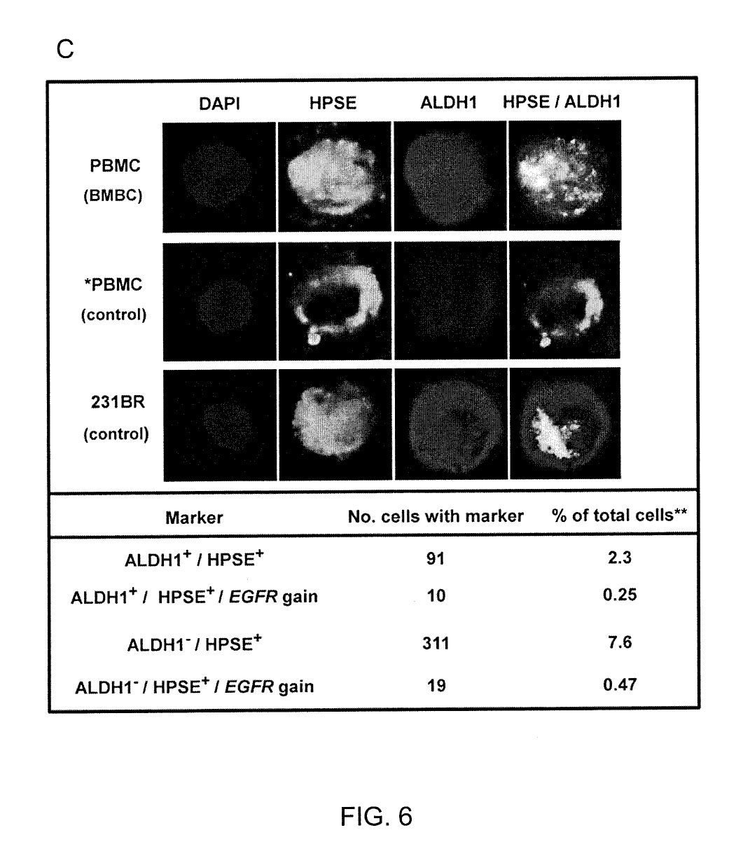

Findings have shown that CTCs derived from breast cancer patients with clinically detectable BMBC rarely express EpCAM, and CellSearch™-undetectable (TC status positively correlate with presence of brain metastasis in a large cohort of patients (Mego et al., 2011). Because the CellSearch™ technology is unable to interrogate the EpCAM-negative CTC subpopulation, the inventors implemented a study design to capture this subset using technologies and platforms alternative to CellSearch™. Initially, the inventors employed a technology, termed FICTION, which is provided by the BioView-Duet™ platform (BioView™ Ltd, Rehovot, Israel). FICTION combines protein detection by immunofluorescence (IF) with gene amplification by fluorescence in situ hybridization (FISH) analyses within the same slide of isolated peripheral blood mononuclear cells (PBMCs). Automated quantification of the signal is then achieved by the BioView™ system to visualize/assess properties of cancer-associated circulating cells according to specific IF/FISH patterns (Katz et al., 2010). It is known that the epidermal growth factor receptor (EGFR) acts as a high-risk predictor for BMBC disease and is an important biomarker for CTCs (Eichler et al., 2011; Rimawi et al., 2010). Accordingly, they examined circulating cells for presence of EGFR amplification in PBMCs isolated from patients with BMBC (clinical diagnosis). Aberrant EGFR amplification was observed in multiple and independent patients' PBMCs (FIG. 6A) whose primary tumors possessed EGFR amplification. Further, by performing rigorous comparisons employing the same patient samples and BioView™ and CellSearch™ platforms in parallel. EGFR-amplified cells could be detected by the former but not the latter, substantiating their EpCAM-negative status. Lastly. EGFR amplification correlated with the expression of heparanase, a potent pro-tumorigenic, pro-angiogenic, and pro-metastatic molecule (FIG. 6B) (Fehm et al., 2010; Zhang et al., 2010; Zhang et al., 2011; Ridgway et al., 2012; Vreys and David, 2007) and aldhehyde dehydrogenase1 (ALDH1), a known tumor-initiating cell marker (FIG. 6C) (Ginestier et al., 2007). The inventors considered CellSearch™-undetectable/EGFR+/HPSE+/ALDH1+ cancer-associated circulating cells (CACCs).

### Example 4

### Selection and Isolation of BMBC-Associated CTCs

As a second step, the inventors considered that breast cancer brain-homing CTCs are present within the CACC subset described above. To capture CTCs, the inventors developed strategies to isolate EpCAM-negative neoplastic cells within the PBMC population of breast cancer patients. First, PBMCs were isolated from copious amounts of blood (35-45 mls) from breast cancer patients, then selected for CD45 negativity but ALDH1 positivity and EpCAM status using multi-parametric flow cytometry. The inventors isolated EpCAM+/ALDH1+/CD45− and EpCAM−/ALDH1+/CD45− subsets from breast cancer patients' blood but not from PBMCs of healthy donors of the same race and age characteristics, and employing the same FACS procedures (Table 3).

TABLE 3
Isolation and sorting of PBMCs from breast cancer patients

| Approach | Population | Patient-1 | Patient-2 | Patient-3 | Control |
|---|---|---|---|---|---|
| Ficoll-Hypaque | PBMCs | 1,131,975 | 1,584,143 | 1,110,556 | 1,057,540 |
| FACS | CD45+ | 637,442 | 839,595 | 621,911 | 1,018,490 |
| | ALDH1+ | 18,109 | 14,257 | 12,216 | 616 |
| | EpCAM+/ALDH1+/CD45− | 227 | 1141 | 36 | 0 |
| | EpCAM−/ALDH1+/CD45− | 971 | 2,042 | 633 | 0 |

* Control: PBMCs from patients without breast cancer.

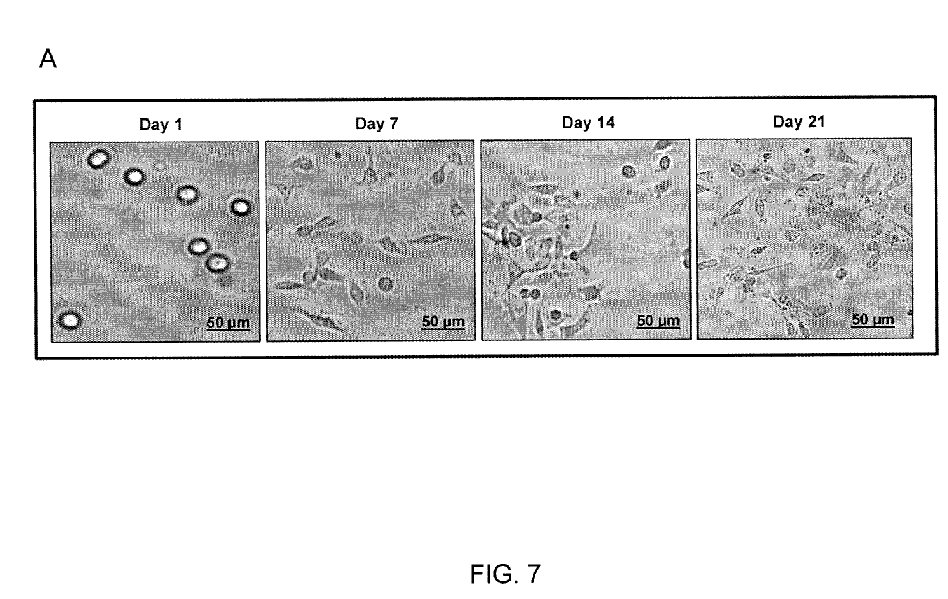

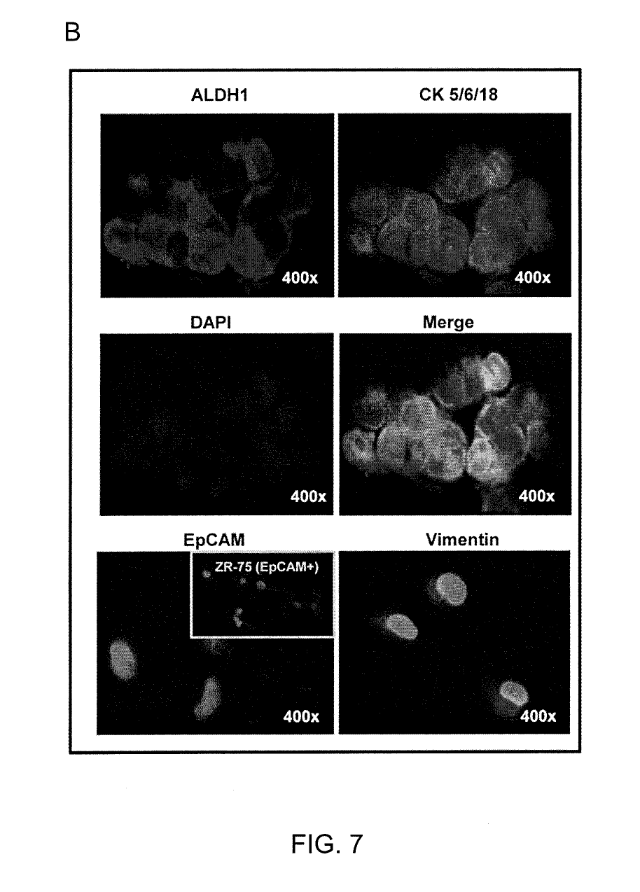

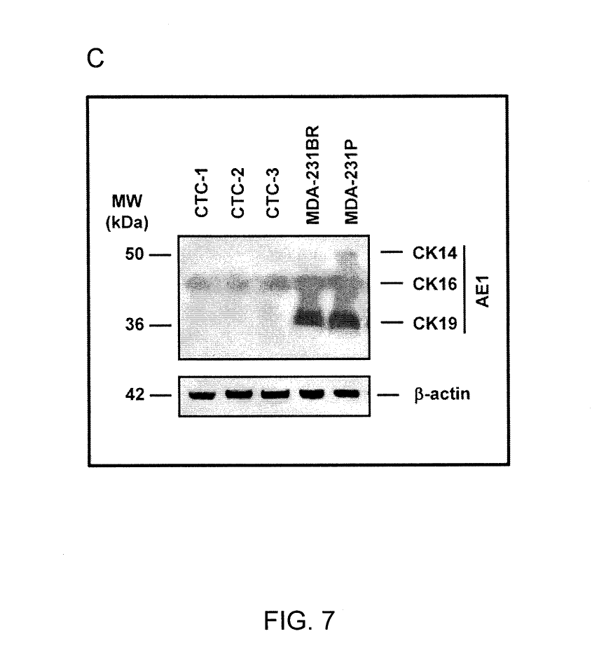

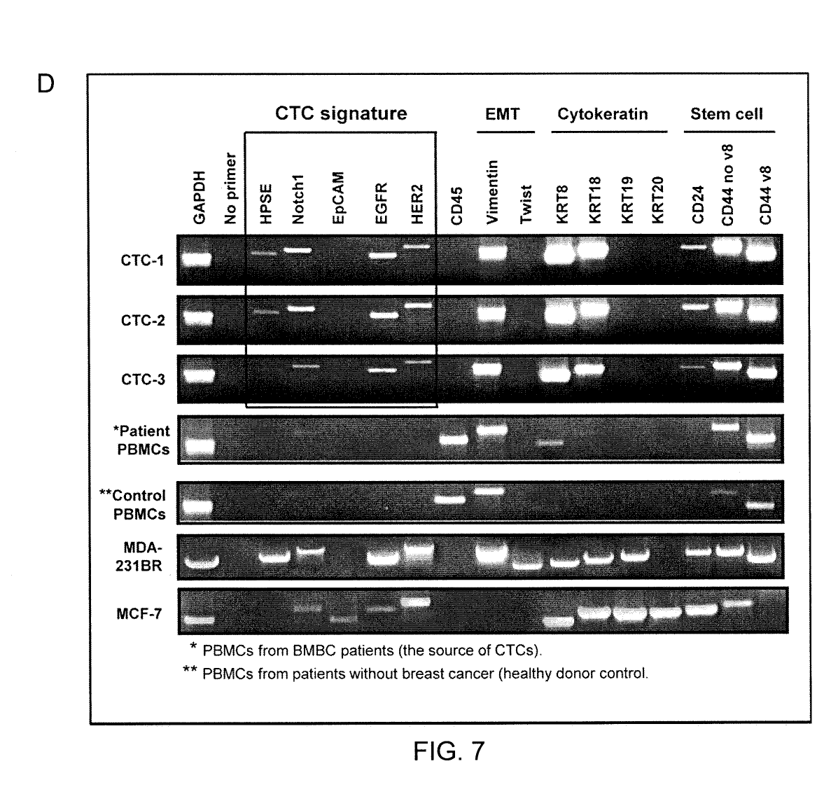

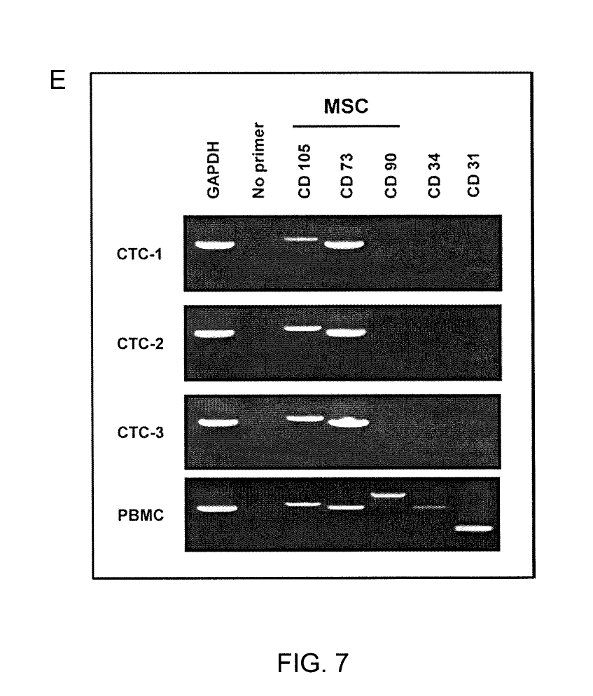

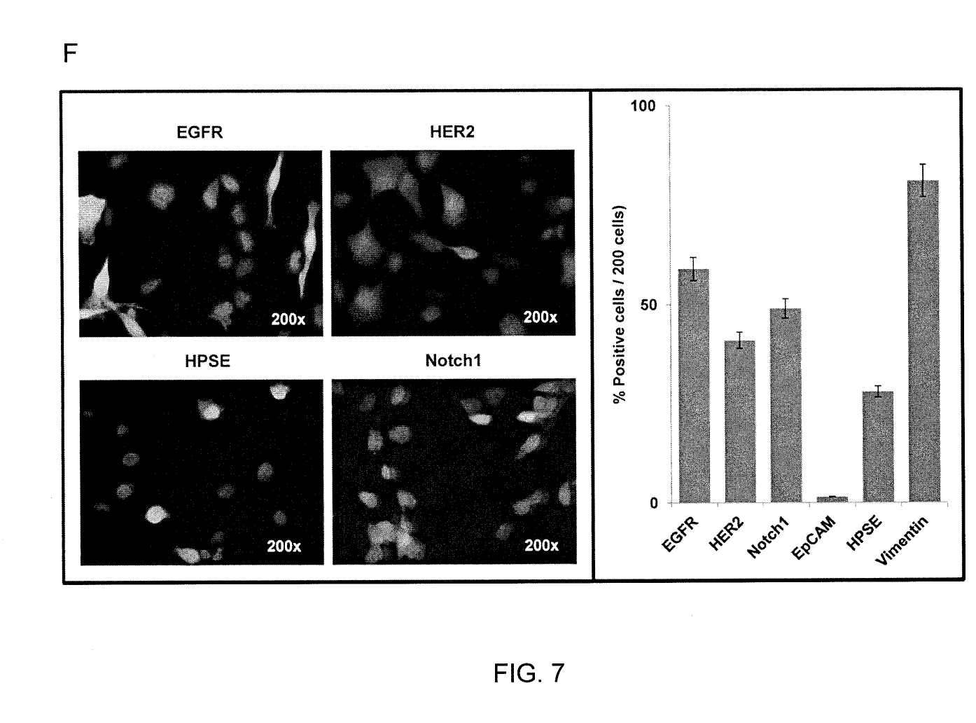

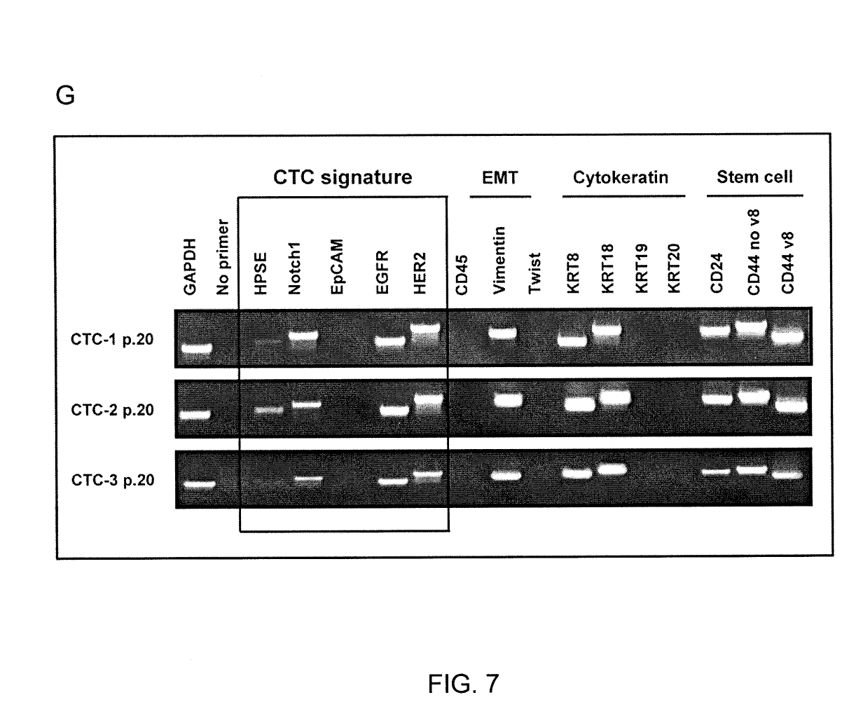

Second, FACS-selected EpCAM+/ALDH1+/CD45− or EpCAM−/ALDH1+/CD45− circulating cell subsets were cultured for further characterization. Cells were cultured in vitro using special media and procedures with their morphology and properties (survival, growth). Considering that captured cells used to survive in blood under a suspension status, the inventors provided a period of transition using stem cell culture medium plus 10% FBS for the first week, then this culture medium was switched to EpiCult-C Basal Medium (Stem Cell Technologies, Durham, N.C.). Cells were carefully monitored for survival and growth, and colonies were initiated starting from a single cell (FIG. 7A). Thirteen colonies were observed in patient-1 by day 21 of cell culture; 7 in patient-2; and 11 in patient-3, respectively (Table 4).

TABLE 4
Summary of FACS-captured cell survival and growth

| | Cell Culture | | No. of clones | | |
|---|---|---|---|---|---|
| Subpopulation | Day | Medium | Patient-1 | Patient-2 | Patient-3 |
| EpCAM⁺/ALDH1⁺/CD45⁻ | 1-7 | Stem M | 0 | 0 | 0 |
| | 8-14 | Epi + 10% F | 11 | 9 | 1 |
| | 15-21 | Epi + 10% F | 3 | 5 | 0 |
| | 22- | Regular M | 0 | 0 | 0 |
| EpCAM⁻/ALDH1⁺/CD45⁻ | 1-7 | Stem M | 0 | 0 | 0 |
| | 8-14 | Epi + 10% F | 4 | 2 | 5 |
| | 15-21 | Epi + 10% F | 13 | 7 | 11 |
| | 22-plus | Regular M | Not count | Not count | Not count |
| | | | (expand culture) | (expand culture) | (expand culture) |

Epi = EpiCul-C Basal medium;
Stem M = Stem cell culture medium.
Regular M = DMEM/F12 + 10% FBS + 1% P.S.
*EpCAM+ and EpCAM− cells fromTable 1 were plated in 96-well cell culture plates, ~10 cells per well.
More than 20 cells per clone developed from a single cell.

When colonies became larger, they were transferred into 24-well or 6-well cell culture plates for expansion, then transferred to T-75 flasks for additional tissue culture expansion and characterization to establish CTC lines—CTC-1, CTC-2, CTC-3 respectively. Surviving cells were examined to confirm expression of ALDH1, contrasting levels of EpCAM, and presence of tumor cell markers, notably cytokeratins (CKs) 5/6/18 and CK 16, latter is a specific marker for breast cancer (Cristofanilli et al., 2004; Pantel et al., 2008; Joosse et al., 2010). Immunofluorescence analyses showed that ALDH1 and CK5/6/18 were highly expressed in EpCAM-negative cells (FIG. 6B). CK16 was also detected by Western blotting and its expression levels were similar to ones of human breast cancer cell lines (MDA-MB-231 parental and the in vivo selected brain metastatic variant MDA-MB-231BR)(Palmieri et al., 2007). However, different patterns were observed between CTCs and MDA-MB-231BR/MB-231 parental cells (FIG. 6C).

### Example 5

### Characterization of CTCs by Thermodynamically-Matched Primer RT-PCR

The inventors considered that the unique gene signature, was present in CTCs derived from metastatic breast cancer patients. To test for the expression and biological relevance of this signature, they developed a specific semi-quantitative RT-PCR assay by designing thermodynamically-matched PCR primer pairs for the parallel detection of multiple markers either selected because of their proven relevance to BMBC (HPSE, Notch1, EGFR, HER2) (Cristofanilli et al., 2004; Pantel et al., 2008; Palmieri et al., 2007; Rimawi et al., 2010; Fehm et al., 2010; Zhang, Sullivan, Suyama, et al., 2010; Zhang, Sullivan, Goodman, 2011; Ridgway et al., 2012; Vreys and David, 2007; Li et al., 2008), or neoplasticity beyond the CellSearch™ CTC definition, or in cell stemness (see below Examples). RT-PCR assays were subsequently performed using these specific primers within the same experimental conditions and in parallel. Additive to analyzing FACS-isolated CTCs from a symptomatic BMBC patient and two non-symptomatic breast cancer patients as template material for the RT-PCR assays, the inventors also utilized PBMCs collected from the same patient which did not undergo FACS selection for CTC isolation; along with PBMCs isolated from healthy subjects of the same race, similar age, and background (controls). These cells were positive for HPSE, Notch1, EGFR, and HER2 transcript expression, however negative for CD45 and, notably, EpCAM (FIG. 7D). Because it has been demonstrated that these genes are critical and highly expressed in metastatic breast cancer patients, the inventors termed them “the CTC signature”. Additive to this signature, the inventors examined other important markers of tumor-initiating cells such as the CD24^{low}/CD44^{high} ratio, and CD44 variants expression (Li et al., 2008). A typical pattern of cancer stem cell—CD24^{low}/CD44^{high} was observed in cultured CTCs, but not in PBMC controls. The average ratio of CD24/CD44 transcript expression in these CTC lines was 0.7. Markers of potential epithelial-mesenchymal transition (EMT, e.g., vimentin and Twist) (Mego et al., 2010) were also examined. Interestingly, higher vimentin expression was observed in cultured CTCs, whereas Twist expression was negligible (Tarin, 2012). Second, there was a robust presence for CK8 and CK18 transcripts (16% and 11% above GAPDH levels, respectively) while ones for CK 19 and CK20 genes were below the threshold detection limit in all CTC samples analyzed. The differential CK transcript levels can be related to the particular stage of tumor progression since it is known that the expression of several CKs changes during metastasis (Joosse et al., 2012). Additional markers of neoplasticity included uPAR, Muc1, and caveolin1, whose transcripts could be detected in CTCs. Third, the inventors examined brain metastatic MDA-MB-231BR (231BR for brevity) and non-metastatic MCF-7 breast cancer cells for the CTC signature (controls). 231BR cells expressed all genes of the CTC signature with patterns and levels similar to CTCs. Conversely, CTC signature-assessed HPSE was nearly absent in MCF-7 breast cancer cells, implying the relevance of HPSE as a critical player in metastasis and BMBC mechanisms (FIG. 7D) (Zhang L, Sullivan P, Suyama et al., 2010; Zhang L X, Sullivan P S, Goodman et al., 2010; Ridgway et al., 2012; Vreys and David. 2007). Fourth, to validate that FACS-captured CTCs did not represent some hematopoietic or non-CTC cell populations, they performed RT-PCR analyses for markers expressed either in circulating endothelial cells (CD 105, CD31), bone marrow hematopoietic cells (CD34), or mesenchymal stem cells (CD 105, CD75, CD90 triplet and the lack for the expression of CD45, CD34, and CD31) (Dominici et al., 2006). The three CTC lines selected from corresponding patients (CTC-1, CTC-2, CTC-3, respectively) were all negative to these non-CTC marker parameter while opposite patterns were detected in PBMCs (FIG. 7E) (Fonsatti et al., 2000; Ostapkowicz et al., 2006). Further, transcript expression for genes of the CTC signature translated to corresponding protein presence by IF analyses which confirmed the absence of EpCAM in these cells (FIG. 7F). Accordingly, FACS-captured cancer-associated circulating cells from patients' PBMCs expressed a set of marker genes and proteins: “the CTC signature”.

### Example 6

### CTC Genotyping Analyses

To validate that CTC lines represent putative CTCs and are not the result of cell cross-contamination, genotyping was performed by short tandem repeat (STR) DNA fingerprinting and data compared to the Characterized Cell Line Core (CCLC) database of MD Anderson Cancer Center (Houston, Tex.), (Table 3).

The three CTC lines possessed STR loci fingerprinting profiles (e.g., D18S51, D7S820, D8S 1179, FGA, etc.) distinct from either established BMBC cell lines (i.e. 231BR, MDA-435), poorly brain metastatic (MDA-MB231 parental) or non-metastatic (MCF-7) breast cancer cells. Second, fingerprinting profiles showed differences (i.e., D21S 11, FGA) among CTC lines suggesting that these lines are distinct although sharing the CTC signature. Third, the neoplastic nature of CTC lines was further validated by the detection of known mutations for hallmark cancer genes (BRCA, KRAS, and TP53) by MALDI TOF Mass Array system (Sequenom Inc., San Diego, Calif.).

Lastly, the inventors confirmed that FACS-captured CTCs maintained the signature in long-term culturing. Gene transcripts for members of the CTC signature were maintained (higher than 20 cell passages) together with other markers of neoplasticity and cell stemness (FIG. 7G).

### Example 7

### Capture of EPCAM-CTCs Over-Expressing EGFR, HER2, and Notch1

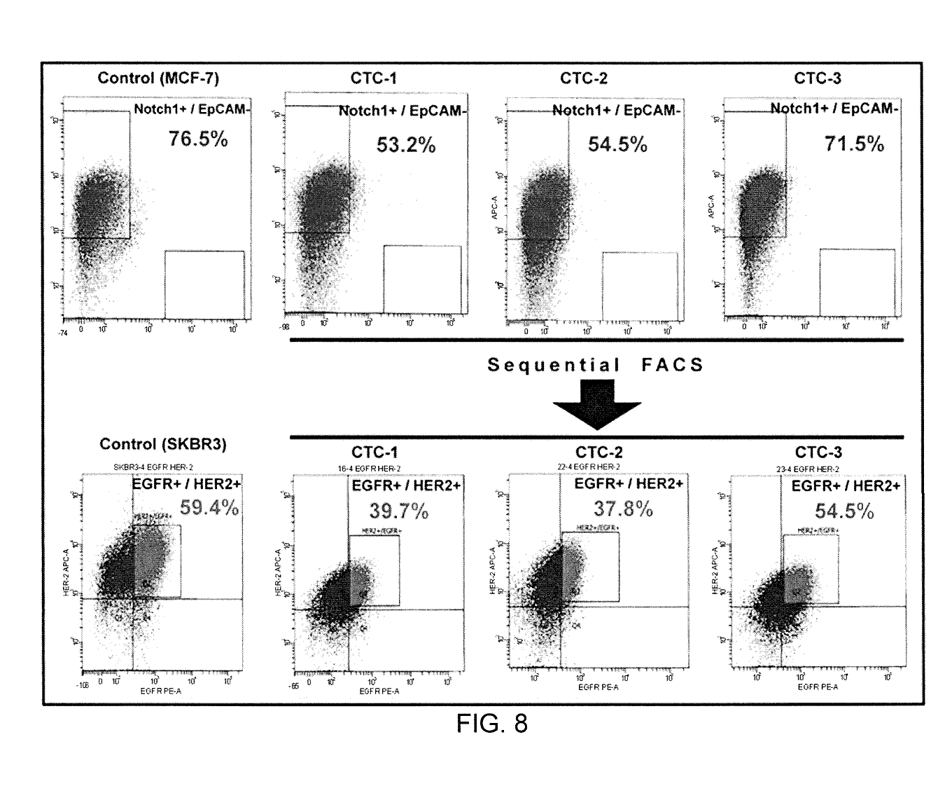

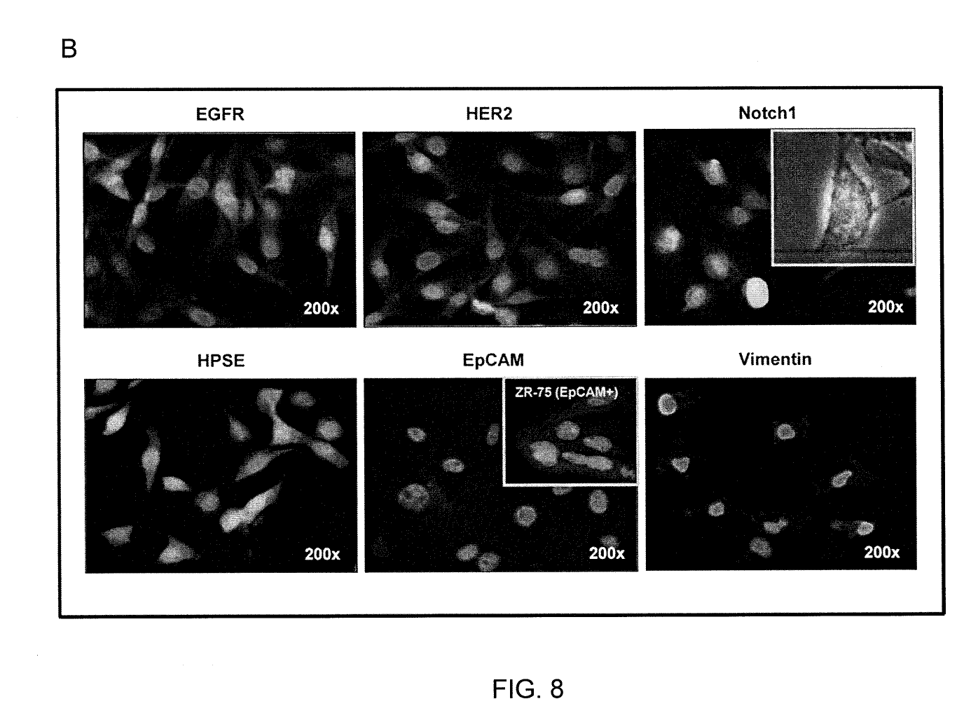

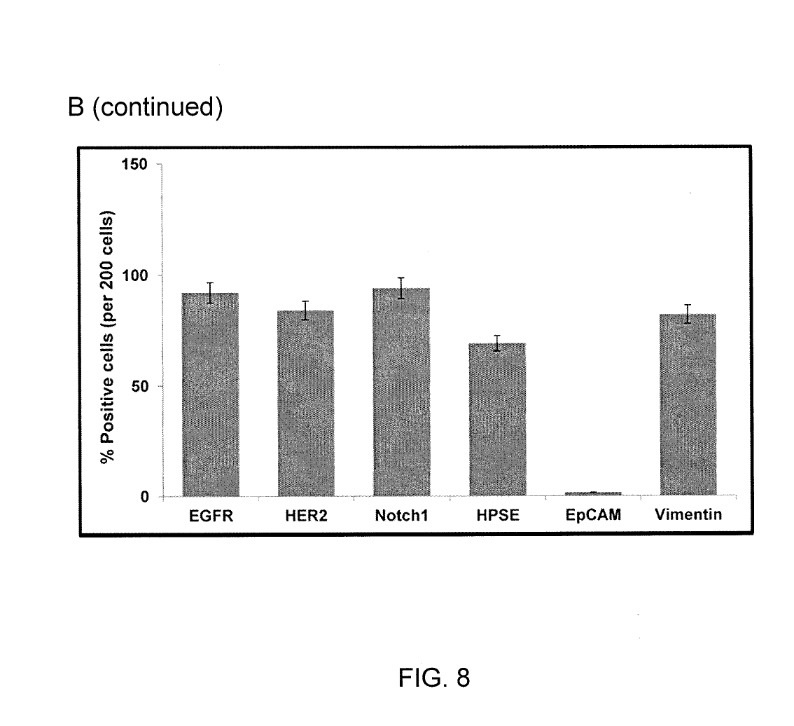

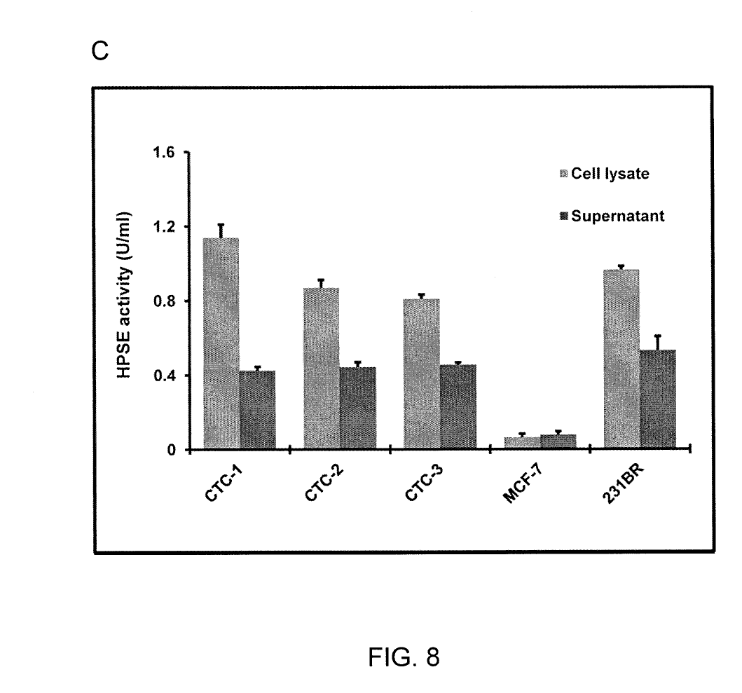

To assess the biological relevance of the CTC signature in events leading to BMBC onset, primary EpCAM-negative CTCs were sequentially sorted using anti-Notch1, anti-HER2 and anti-EGFR. The proportion of total viable EpCAM-negative population selected for Notch1 over-expression was 53.2%, 54.5%, 71.5% for CTC-1. CTC-2, CTC-3, respectively (FIG. 8A, upper panels). Selected EpCAM-negative but Notch1 over-expressing cells were expanded and sorted further to obtain HER2 and EGFR CTC over-expressors. Sorting in the presence of specific antibodies to HER2 and EGFR resulted in the selection of CTCs possessing both HER2 and EGFR over-expression and represented 39.7% for CTC-1, 37.8% for CTC-2, 54.5% for CTC-3 populations, respectively (FIG. 8A, lower panels). High EGFR, HER2, and Notch1 protein expression in these cells was confirmed by IF staining experiments using specific antibodies against these markers. However, and similar to primary CTC subsets, CTC over-expressors maintained the EpCAM negativity status (FIG. 8B). Further, the expression and activity of HPSE as a key player of the CTC signature were examined since this molecule is expressed/over-expressed at higher levels in the cytoplasmic compartment. The inventors detected presence of HPSE activity in both cell lysates and supernatants of the (CC cultures (FIGS. 8B, 8C). CTC-1 expressed highest HPSE activity levels in cell lysate while activity in CTC-2 and CTC-3 lysates approximated ones of 231BR cells (control). Conversely, levels of HPSE activity were similar in supernatants of all three CTC lines. Captured CTCs over-expressing Notch1/EGFR/HER2/HPSE were however negative to EpCAM and have been termed “CTC over-expressors” (CTC-ov).

### Example 8

### CTC Over-Expressors are Invasive and Brain Metastasis-Competent

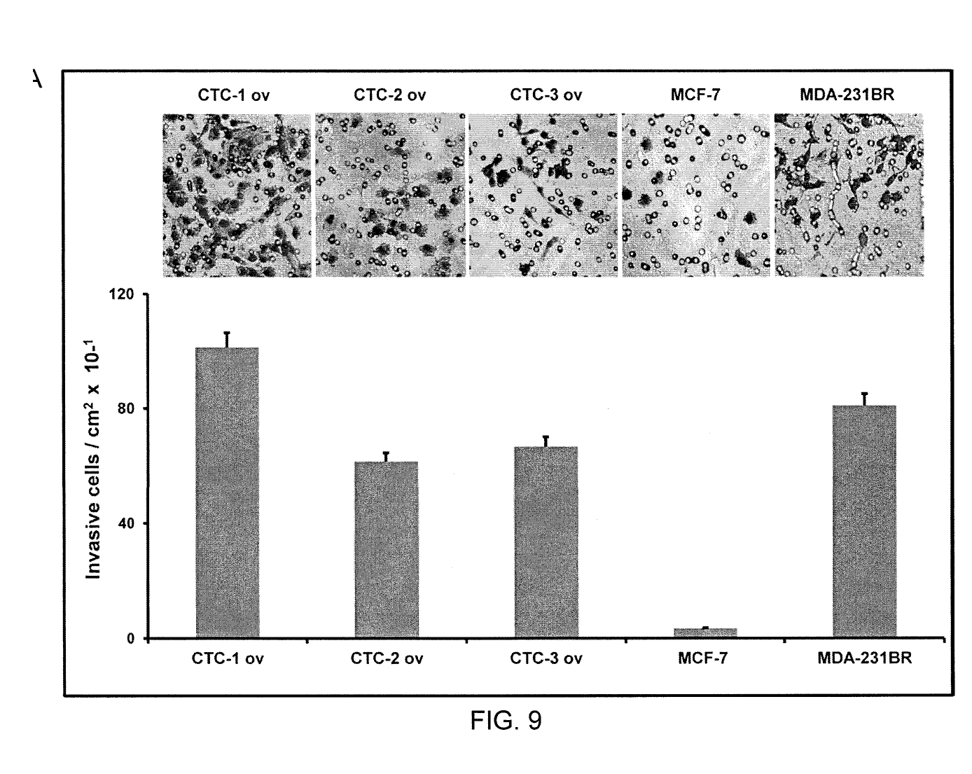

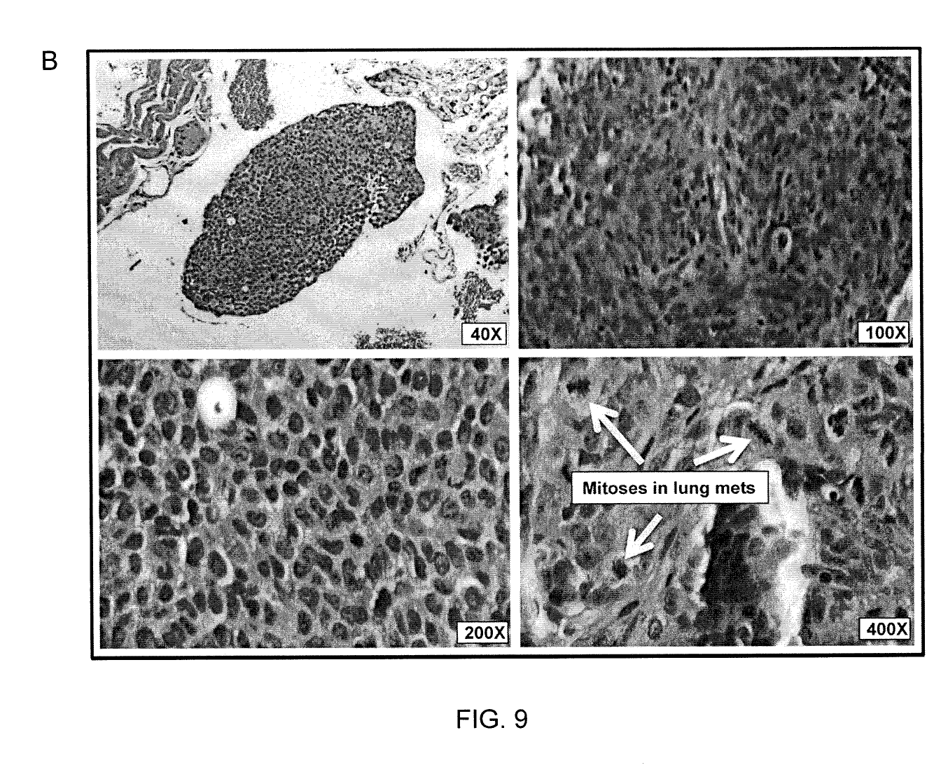

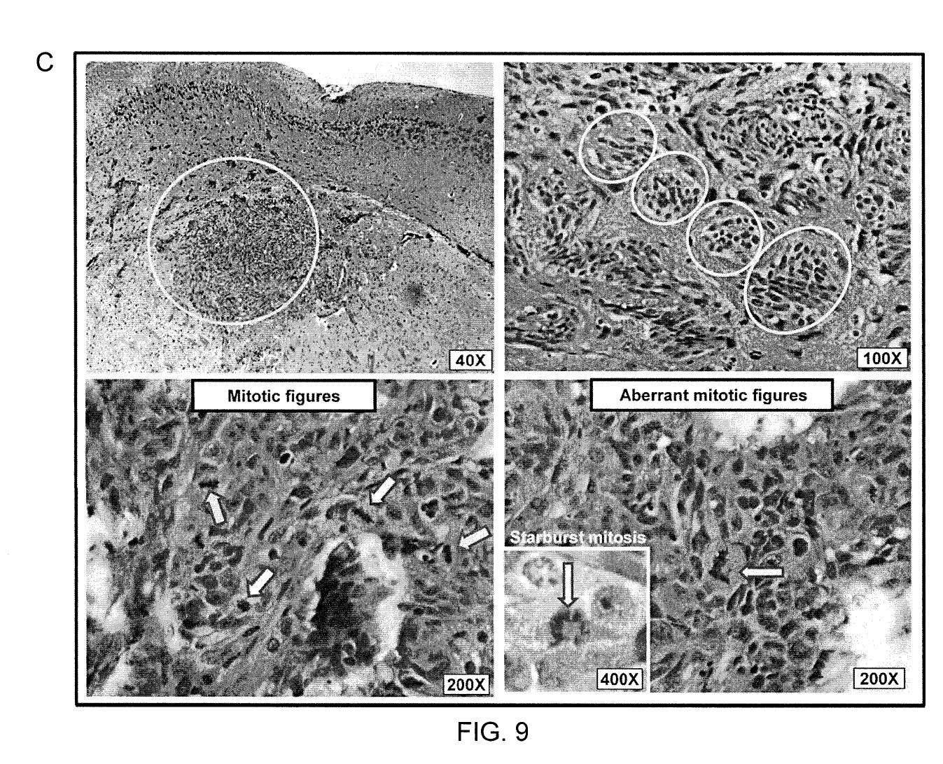

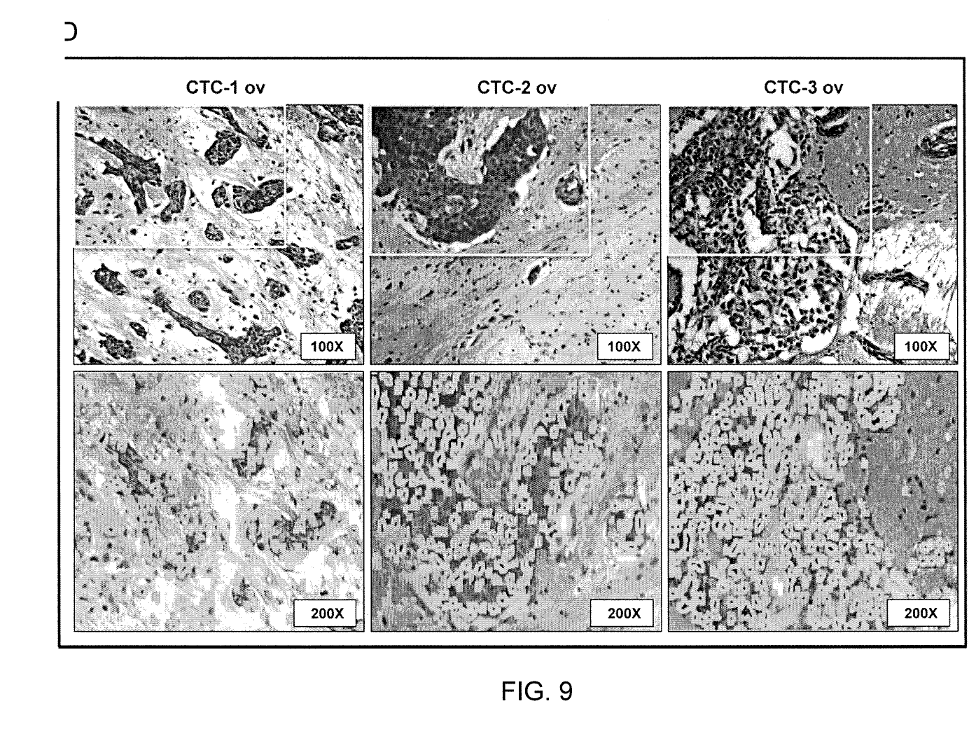

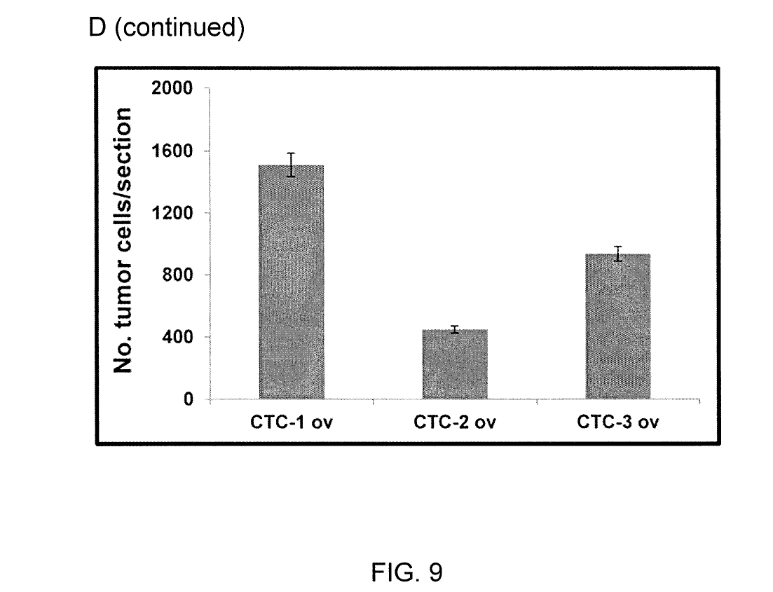

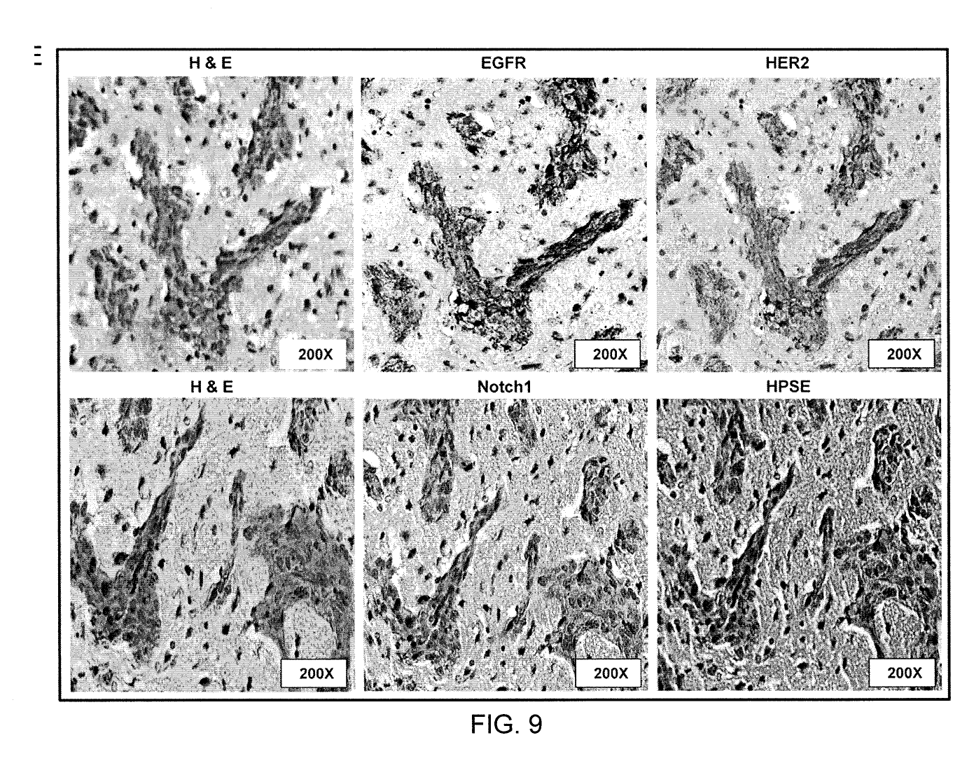

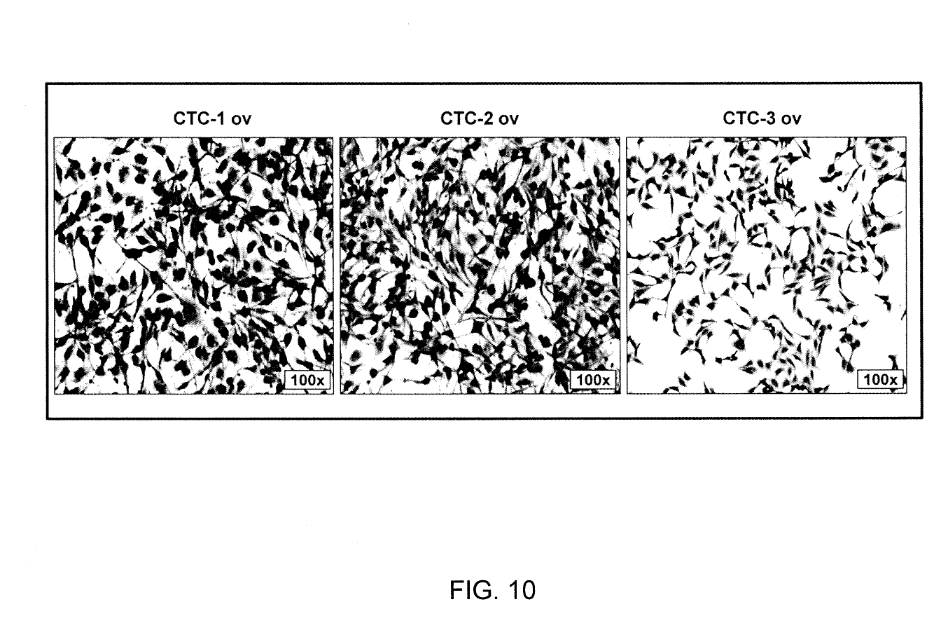

To evaluate invasive and metastatic functionalities of selected CTC over-expressors, the inventors examined whether these CTCs possessed invasive capabilities. Accordingly, the inventors performed in vitro chemoinvasion assays (Matrigel™ chambers) using CTC over-expressors and assessed their invasive abilities, and compared to the highly invasive and brain metastatic MDA-MB-231 BR (Katz et al., 2010; Hirose et al., 2010) and to tumorigenic but poorly invasive MCF-7 breast cancer cells (Sieuwerts et al., 2009; Königsberg et al., 2011). Data showed that CTC over-expressors were highly invasive, e.g., CTC-1 possessed highest invasive abilities that was approximately 25% higher than MB-231BR cells (p<0.05) (FIG. 9A). Importantly, to examine whether CTC over-expressors were capable of generating tumors, the inventors injected them either intracardiacally or tail vein in immunodeficient animals (nu/nu mice; 5×10⁵ cells/mouse; n=20) monitoring for metastasis formation. All CTC over-expressors had abilities to metastasize to lungs and brains by four-six weeks after injection in animals, and to generate macro-metastatic and micro-metastatic lesions (FIGS. 9B-9D).

Of note, normal and aberrant mitotic figures could be identified as hallmarks of cell proliferation and neoplastic behavior, e.g., starbust mitosis (FIG. 9B). Compared to primary CTCs, CTC over-expressors had a significantly increased incidence to colonize brain. Incidence of brain metastasis increased from 20% to 80% for CTC-lov, and from 0 to 60% for CTC-2ov and CTC-3ov, respectively. CTC-induced breast cancer brain metastasis also presented a typical branching pattern of tumor growth and presence of micro- and macro-metastasis (FIG. 9C). Further, CTCs-induced brain metastases were evaluated at a single tumor cell level by the Cri Vectra Intelligent™ automated slide analysis system (Cambridge Research & Instrumentation Inc., Boston, Mass.) (FIG. 9D). Lastly, to evaluate whether the expression of the (CC signature was present in experimental brain metastasis, the inventors examined the expression of signature proteins in mouse brain tumors by immunohistochemistry. Brain tumor tissues displayed the presence of proteins of the CTC signature (FIG. 9E). Collectively, the inventors demonstrate that CTCs can form metastasis in xenografts, and that the presence of the CTC signature is necessary to promote BMBC.

### Example 9

### Significance of Certain Embodiments of the Invention

Circulating tumor cells represent the “seeds” of metastasis and a promising alternative to tumor biopsies to detect, investigate, and monitor solid tumors: enumerating CTCs has been shown to act as an independent prognostic indicator of tumor progression with a high therapeutic value (Cristofanilli et al., 2004; Pantel et al. 2008; Mego et al., 2011; Stott et al., 2010: Nagrath et al., 2007; Pecot et al., 2011; Sieuwerts et al., 2009; Maheswaran et al., 2008). Thus far, only one CTC platform—CellSearch™ (Veridex, LLC)— has been cleared by the US Federal Drug Administration for clinical CTC testing. It consists in capturing CTCs which are positive for EpCAM and cytokeratins but negative for CD45, a marker of normal hematolymphoid cells (Cristofanilli et al., 2004; Pantel et al., 2008). Additional CTC platforms have been developed and used in successful studies, however with limitations of analyzing only one subset of CTCs, e.g., EpCAM-positive CTCs (Pantel et al., 2008; Mego et al., 2011; Stott et al., 2010; Nagrath et al., 2007; Pecot et al., 2011). Key purpose of this study was to develop novel approaches to detect, identify, and characterize EpCAM-negative CTCs present in breast cancer patients. Many investigations have described multiple methods for CTC detection and enrichment which are usually based on density gradient centrifugation, filtration or immunomagnetic procedures. These sensitive technologies are able to identify CTCs at a frequency of 1 per 10⁶-10⁷ nucleated blood or bone marrow cells (Pantel et al., 2008), however, they can be limiting because of the heterogeneous nature of CTCs, especially in relation to EpCAM-negative CTCs (Sieuwerts et al., 2009; Königsberg et al., 2011).

The inventors considered that the assessment of a multiplexed biomarker could provide an approach for the identification of a specific organ-targeting CTC subset (i.e, brain-homing CTCs with undetectable/negative EpCAM) because of the heterogeneous nature of CTCs which reflect the known heterogeneity of primary and metastatic tumors. The inventors designed investigations to implicate multiple and complementary technologies to detect and analyze the EpCAM-negative breast cancer CTC subpopulation that could not be captured by CellSearch™ (undetectable CTCs). First, the inventors characterized CTCs in blood samples of BMBC patients using combinations of FICTION/BioView™ and CellSearch™; followed by sorting PBMCs from BMBC patients for cancer-associated circulating cells, and characterizing these cells as CTCs. Further sorting allows one to obtain EpCAM-negative, Notch1/EGFR/HER2CTCs over-expressors. Finally, the inventors interrogated CTC over-expressors and demonstrated that these cells are highly aggressive in xenografts and brain metastasis-competent.

For the initial selection for CTCs, the tumor-initiating cell (cancer stem cell) marker ALDH1 was chosen since breast cancer cells expressing ALDH1 are capable of generating in vitro mammospheres as well as duct formation, and promoting oncogenesis in experimental animals (Ginestier et al., 2007). Conversely, cells being ALDH1-negative, and/or CD44+/CD24−/lin-, could not form tumors when transplanted into the mammary fat pad of nude mice. Accordingly, the inventors applied ALDH1+/CD45−/EpCAM+/− as selection marker for possible CTCs by sorting PBMCs of breast cancer patients. However, the inventors could not exclude having captured normal stem cells since they also express ALDH1 (Ginestier et al., 2007). Because these cells (“primary CTCs”) possessed low or no abilities to induce brain metastasis (0-20% BMBC frequency), they sorted CTCs employing antibodies to Notch1, EGFR, and HER2, respectively (McGowan et al., 2011; Hirose et al., 2010; Palmieri et al., 2007; Rimawi et al., 2010; Fehm et al., 2010). Data from animal model demonstrated that FACS-captured cells positive for these markers (“CTC over-expressors”) increased the incidence of brain metastasis in mice to 100%. These results indicate that the CTC signature can predict BMBC development.

Notch1 plays roles in cancer progression and is commonly expressed in aggressive breast cancer subtypes. Several investigations have demonstrated that Notch signaling inhibition prevented the colonization of human MDA-MB-231BR cells in the brain and inhibited breast cancer brain metastasis (McGowan et al., 2011; Hirose et al., 2010); and that Notch1 works in synergy with HER2 and/or EGFR (Hirose et al., 2010). Previous studies have also demonstrated that the expression and/or amplification of EGFR and HER2 genes can directly distinguish tumor cells from non-malignant epithelial cells or leukocytes. Specifically, BMBCs frequently possess EGFR and/or HER2 over-expression (Eichler et al., 2011; Palmieri et al., 2007; Rimawi et al., 2010). Therefore, the inventors sorted CTCs for Notch1 positivity which was followed by the selection of EGFR+ and HER2+ CTCs. The inventors found that CTC-1 cells derived from a BMBC patient (triple-negative) expressed EGFR and HER2 at both mRNA and protein levels (FIGS. 7D and 8B). This further demonstrates that HER2 status is altered from the primary tumor to CTCs and aligns well with similar findings (Sieuwerts et al., 2009; Fehm et al., 2010). Thus, there is provided further evidence that CTCs develop a differential HER2 content over the course of neoplastic progression, in at least certain aspects of the invention.

Heparanase (HPSE) is another component of the BMBC CTC signature and a potent pro-tumorigenic, pro-angiogenic, and pro-metastatic molecule, initiating multiple effects which drastically alter the metastatic outcome (Fehm et al., 2010; Zhangm Sullivan, Suyama, et al., 2010 Zhang L X. Sullivan P S, Goodman, et al., 2011; Ridgway et al., 2012; Vreys and David, 2007). An established role of heparanase is to release growth and angiogenic factors which avidly bind HS chains of HS proteoglycans as storage depots within the extracellular matrix, thus regulating their overall levels and biological potency (Fehm et al., 2010; Zhangm Sullivan, Suyama, et al., 2010; Zhang L X, Sullivan P S, Goodman, et al., 2011; Ridgway et al., 2012; Vreys and David, 2007). Of note, highest levels of HPSE activity have been consistently detected in cells selected in vivo for highest propensities to colonize the brain, regardless of the cancer type or model system used (Zhang L X, Sullivan P S, Goodman, et al., 2011). Recent findings have also demonstrated that HPSE has functions which are independent of its enzymatic activity and mediated by its latent form, e.g., promoting cell adhesion, augmenting EGFR phosphorylation, and acting as a signal transducer (Ridgway et al., 2012; Cohen-Kaplan et al., 2008). The therapeutic disruption of heparanase thus provides an opportunity to block multiple pathways that control tumor-host interactions and are crucial for tumor cell adhesion, growth, and metastatic onset, particularly to brain. The data indicate that HPSE expression correlates with EGFR amplification and ALDH1 positivity. The inventors consider in specific embodiments that HPSE expression is central in BMBC, e.g., in the initial events of brain metastasis and cross-talk between CTCs and the brain vasculature (Ridgway et al., 2012).

Of relevance, the inventors have successfully detected, isolated, and established human CTC lines. The inventors validated their metastatic competence in a mouse model. The injection of animals with three putative CTC lines ((CTC-1, CTC-2, CTC-3) captured by ALDH1+/CD45−/EpCAM− sorting and analyzed for the BMBC signature (RT-PCR/IF) resulted in a variability towards the BMBC phenotype: only CTC-1 promoted brain metastasis while no brain metastasis was observed in CTC-2 and CTC-3 lines. In one aspect, the CTC-1 line was derived from a triple-negative BMBC patient, the most aggressive cancer subtype (Harrell et al., 2012). Notably, all three CTC lines could induce BMBC following sorting for Notch1, EGFR and HER2 over-expression. This indicates that the selection of these markers is critical for CTC-primed BMBC onset. It is to be considered that the inventors collected both EpCAM+ and EpCAM− CTCs following first-step FACS analyses characterizing CTCs; however, EpCAM-positive cells could only survive for short periods of time (approximately 2 weeks in culture). Conversely, captured EpCAM-negative cells were able to grow into colonies allowing the development of CTC lines. Genes of the CTC signature affect CTC survival and proliferation, in certain embodiments of the invention, although each gene may play distinct roles in particular aspects of the invention. One can characterize the extent by which biomarker(s) of the CTC signature mediate cell survival and other biological functions using routine methods in the art.

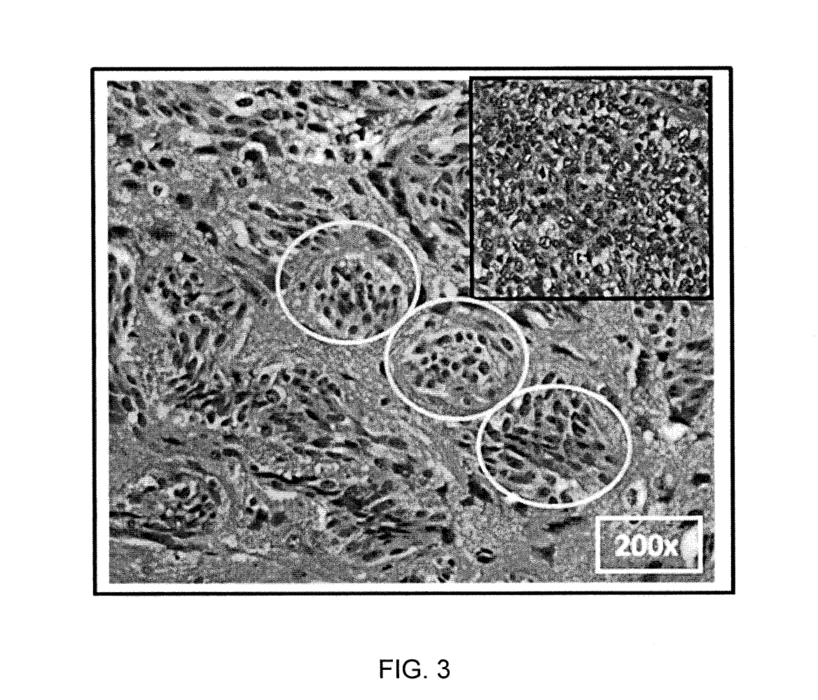

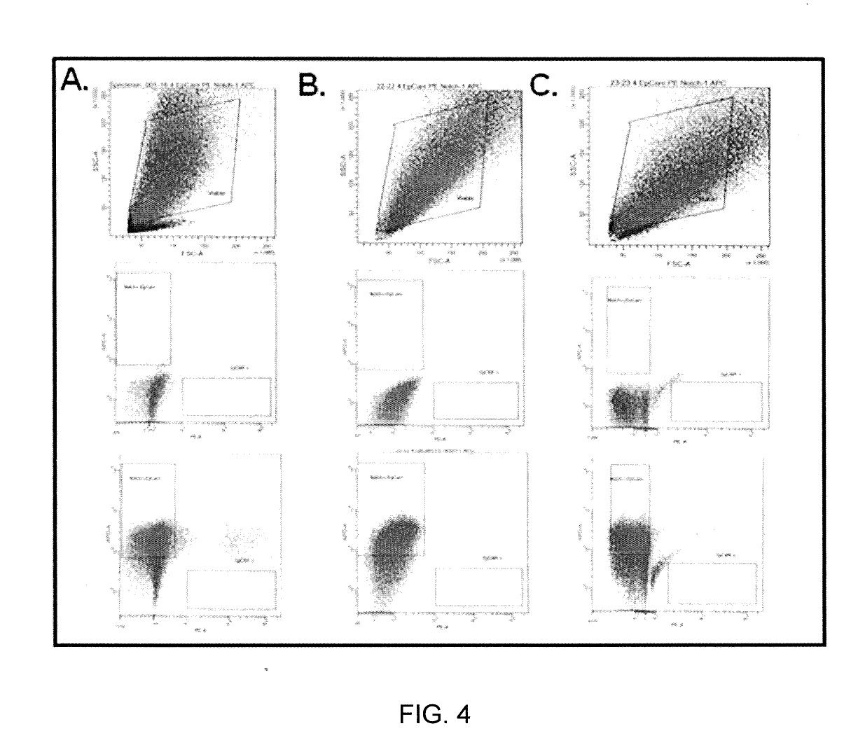

The inventors could not detect brain metastases in a number of animals after the injection of CTCs. Mechanisms for this discrepancy may include: 1) tumor cell dormancy and/or presence of occult brain metastasis which might be at play in these animals without detectable brain metastasis (CTCs populating vs. seeding the brain)(Bos et al., 2009); 2) the cellular localization of the signature proteins might be different in these CTC lines although these CTCs share the same signature. For example, Notch1 mostly localizes at the cell membrane while HPSE resides in the cytoplasm fraction, and Notch1 and HPSE can be present in nuclei of a CTC subset (FIG. 3B). HPSE in nuclear/nucleolar fraction enhanced tumor cells proliferation by directly regulating heparan sulfate-binding DNA Topoisomerase I (Zhang L, Sullivan P, Suyama et al., 2010), but it is unknown whether this localization affects metastasis. Second, since the restriction of the resource, it is difficult to verify whether the primary breast cancers expressed the signature genes (HER2/EGFR/Notch1/HPSE) and ALDH1, and whether the primary breast cancers match their respective CTC lines in STR fingerprinting. Third, it is unknown whether those CTCs without brain metastasis homed at different sites (liver, bone marrow, etc.). One could test whether knocking down genes of the CTC signature will significantly affect CTC-induced breast cancer brain metastasis, either alone or in combination to demonstrate its sufficiency in addition to necessity. These investigations are currently being pursued.

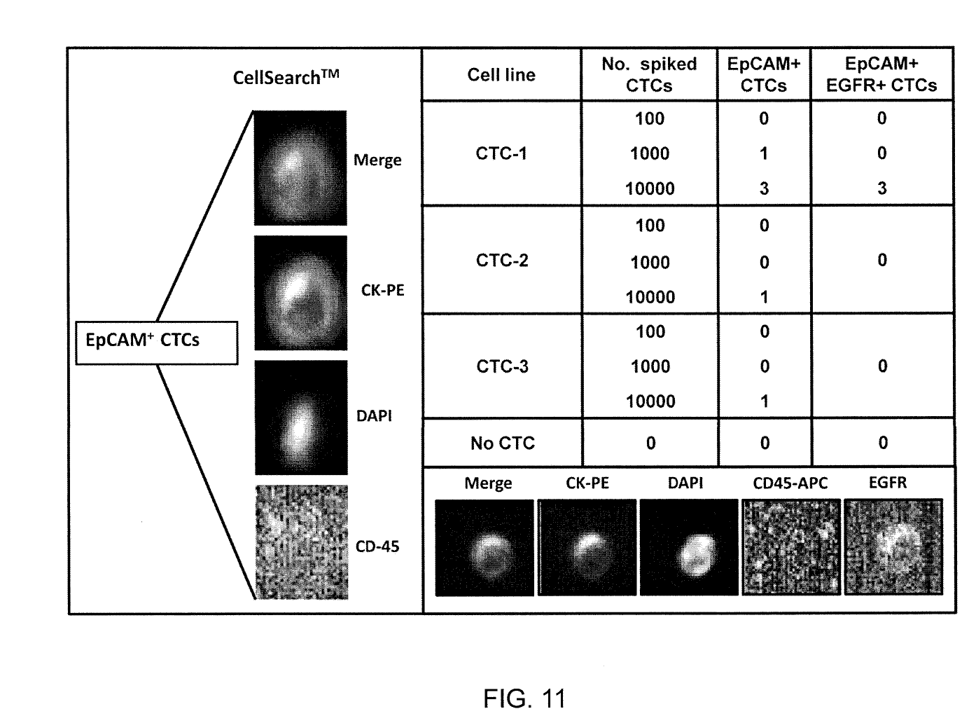

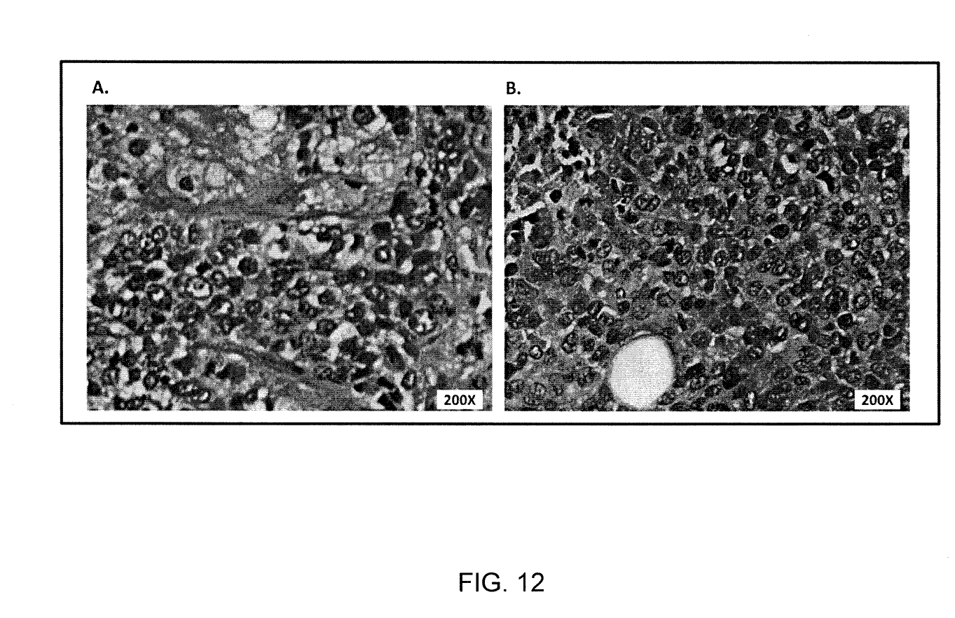

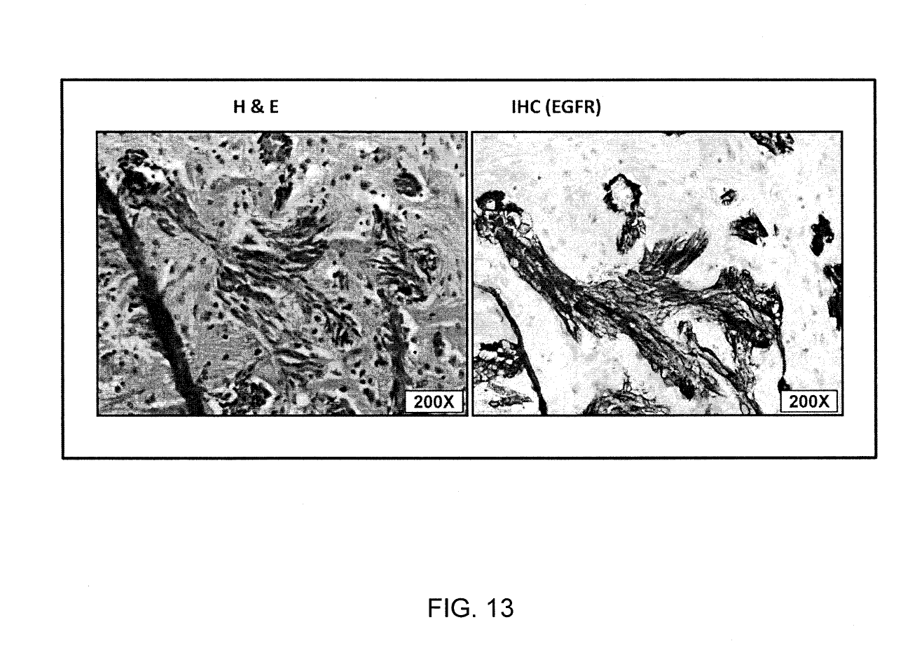

In summary, the results indicate that: (1) CTCs can be detected and isolated by multiparametric flow cytometry from blood of breast cancer patients and cultured in vitro to generate CTC lines; (2) these CTCs have a unique signature (Notch1+/EGFR+/HER2+/HPSE+) but are negative for EpCAM; (3) the development of lung and brain metastases resulted from injecting CTC over-expressors into mice, and CTC-induced metastasis; and possessed morphologies similar to their counterparts in patients' pathological tissues (FIG. 12); (4) Signature proteins were detectable in CTC-induced brain metastasis. These advances are of significance because it is now possible to delineate multiple aspects connected with the complex biology of cancer metastases, including the identification of novel therapeutic targets and the discovery and validation of biomarkers for improved treatment efficacy predicting BMBC and/or preventing secondary metastatic spread. The findings can therefore be highly impactful and with a direct clinical relevance in cancer prevention and therapy scenarios, combating cancer metastasis in general, BMBC in particular.

### Example 10

### Exemplary Methods and Methods

Patient Samples and Blood Collection

Thirty-eight patient samples were collected according to an Institutional Review Board-approved protocol and patient informed consent. Peripheral blood volumes (20-45 mls) were collected in CellSave™ tubes (Veridex, LLC) in sterile conditions. Blood was obtained at the middle of vein puncture after the first 5 mls of blood were discarded to avoid contamination of blood sample with epithelial cells derived from the skin during blood collection. All samples were provided immediately to the laboratory for CTC analyses via pathology courier. The study was conducted using M.D. Anderson Cancer Center medical records database. Only patients starting a new line of therapy were included to the study and patients with concurrent disease were excluded. Patients were required to have clinical and radiological evidence of metastatic progressive breast cancer and underwent systemic therapy as appropriate for their malignancy irrespective of CTC status. Patient data regarding age, tumor histology, hormone receptor and HER2 status, type and number of metastatic sites and systemic therapy were recorded. Peripheral blood mononuclear cells (PBMCs) were isolated by Ficoll-Hypaque gradients as described (Katz et al., 2010). PBMCs were obtained and used for FACS analyses or FISH/IF determinations following cytospins and slide preparation (Katz et al., 2010).

CTC Selection by FACS

Isolated patient PBMCs were analyzed and sorted using the BD FACS Aria II 3 Laser High-speed Sorting Flow Cytometer (Becton Dickinson Inc., San Jose, Calif.) equipped with multiple independent fluorescent channel capabilities and DIVA acquisition software (multiparametric flow cytometry). Each patient PBMC staining set included single-color controls to facilitate rigorous instrument set-up and compensation. A minimum of 5.0×10⁵ up to 2.0×10⁶ events were collected per list mode data file. For the primary selection, markers used for FACS were ALDH1, EpCAM, and CD45. The collected cells were divided into two groups according to EpCAM content: ALDH1+/CD45−/EpCAM+ or ALDH1+/CD45−/EpCAM−. The following reagents and antibodies were used for flow cytometry and cell sorting: mouse anti-human CD45-APC-H7 (BD Bioscience, cat #560274, 10 μl/sample), mouse anti-human EpCAM-PE (eBiosciences, cat #12-9326-71, 20 μl/sample). The ALDEFLUOR assay and kit (StemCell Technologies, Durham, N.C.) was used to isolate the population with a high ALDH enzymatic activity (Ginestier et al., 2007). Cells were prepared for cell sorting by first separating ˜2×10⁶ PMBC cells, then staining with ALDEFLUOR reagent with or without diethylaminobenzaldehyde (DEAB) inhibitor for 1 hour at 37° C. Samples were then centrifuged at 250×g for 5 mins, and suspended in 10 μl ALDEFLUOR buffer (Ginestier et al., 2007). Cells were blocked with 20 μl human Fe receptor inhibitor (eBiosciences, cat #14-9161-73) for 20 min on ice. Following blocking, cells were stained with primary HPSE antibody for 20 min on ice, then washed with 1 ml wash buffer (PBS+1% BSA), and centrifuged at 250×g for 5 min. Pellets were re-suspended in 10 μl wash buffer, then stained with secondary Cy5.5-PE and directly conjugated CD45 and EpCAM antibodies for 20 min on ice. Following another wash, samples were suspended in 0.5 mls wash buffer and analyzed by flow cytometry. Cells were sorted for CD45−, ALDH1+ status, then for EpCAM+ or − status. For experiments utilizing Notch1 sorting, cells were stained using rabbit anti-human Notch1 (Cell Signaling, cat #4380S, 1:50 dilution), then with goat anti-rabbit PE-Cy7 (Santa Cruz, cat #sc-3845, 1:50 dilution). Cells were collected in 0.5 mls RPMI media (Invitrogen) and used for culturing or other experiments.

CTC and Tissue Culture

Cancer-associated circulating cells (CACCs) as potential CTCs were collected from FACS using CD45+ and ALDH1+ markers and cultured in stem cell culture medium (DMEM/F12 containing 5 mg/ml insulin, 0.5 mg/ml hydrocortisone. 2% B27, 20 ng/ml EGF and 20 ng/ml FGF-2) for the first seven days, then switched to EpiCult-C medium from day 8 (StemCell Technologies Inc., Vancouver, Canada) plus 10% FBS, 1% penicillin, at 37° C., 5% CO₂, and continued to grow in this medium. Approximately 0.001% of EpCAM+ CACCs were collected following sequential FACS and 0.0002% were EpCAM-negative CTCs, per characterization using specific CTC markers (FIGS. 2B and 3B). Single colonies were observed under microscope, and were transferred into 24-well (or 6-well) cell culture plates for further growth, and subsequently into T75 tissue culture flasks for additional culture expansion. Human MDA-MB-231 parental and the brain metastasis-selected MDA-MB-231BR cell variant were obtained from Dr. Patricia Steeg (Women's Cancer Section, National Cancer Institute, N.I.H. Bethesda. MD). The 231BR clone is the result of six sequential cycles of intracardial injection of 231 parental cells in nude mice for increased propensities to form brain metastasis in these animals (McGowan et al., 2011; Palmieri et al., 2007). CTC clones were obtained at early passage, DNA fingerprinted, and analyzed for somatic mutations content (e.g., homozygous for TP53 G839A, heterozygous for KRAS G38A and BRAF G1391T) (Ikediobi et al., 2006), and tested for continued and consistent in vivo abilities to metastasize to brain (2011/2012) Cells were cultured in Dulbecco's Modified Eagle Medium plus F12 (DMEM/F12) (Invitrogen, Carlsbad, Calif.) supplemented with 10% fetal bovine serum (FBS) (Invitrogen). All other cell lines used in this study were obtained from the American Tissue Culture Collection (ATCC, Manassas, Va.), DNA fingerprinted, and cultured under prescribed conditions. Cells were harvested using trypsin/EDTA (Cat. #R001100; GIBCO, Grand Island, N.Y.) for spiking experiments using blood from healthy donors (disease-free) or for IF and/or flow cytometry. CTC cultures were DNA fingerprinted to ensure tumor cell fidelity and grown to generate “primary CTCs” (CTC-1, CTC-2, and CTC-3) lines which were used for analyses at early passage. Blood and cell culture DNA Midi kit (cat. #13343, Qiagen, Inc., Valencia, Calif.) was used to isolate DNA from the various cell sources. Cells were grown in DMEM/F12 supplemented with 10% FBS (Invitrogen, Inc.) in humidified, 5% CO2 atmosphere at 37° C., and were assessed as pathogen-free by periodic testing for Mycoplasma contamination. They were employed only at low passage and if Mycoplasma negative.

CTC Genotyping

CTC lines were validated by STR DNA fingerprinting using the AmpF_STR Identifiler kit according to manufacturer's instructions (Applied Biosystems cat 4322288). The STR profiles of CTCs were compared to 231 parental and 231-BR (BMBC) fingerprints, and to the Cell Line Integrated Molecular Authentication database (CLIMA) version 0.1.200808 (http://bioinformatics.istge.it/clima/) (Nucleic Acids Research 37:D925-D932 PMCID: PMC2686526). The STR profiles of CTCs were distinct in eight of the sixteen loci analyzed from known DNA fingerprinting profiles of MB-231 parental and MB-231BR cells. Further, mutation patterns were determined using the Sequenom MALDI TOF Mass Array system that can detect over 100 different common somatic mutations responsible for transformation of normal cells into tumor cells.

RT-PCR

Total RNA from peripheral blood mononuclear cells (PBMCs) was isolated using the RNeasy Plus Mini Kit with QIAshredder (Qiagen, Valencia, Calif.) according to manufacturer's instructions. For each sample, 1 μg total RNA was digested with DNaseI (Invitrogen, Carlsbad. CA) per manufacturer's instructions in a final volume of 11 μl. The reverse transcriptase reaction was accomplished using a Super Script First Strand Synthesis kit (Invitrogen) with 4 μl of the DNaseI digest reaction, which was immediately diluted 1:1 with ice-cold RNAse-free double-distilled water for a final volume of 80 μl. Each PCR reaction used 2 μl of first-strand reaction. The final volume of each PCR reaction was 20 μl. The reaction mix had a final concentration of 1× Amp Gold buffer (Invitrogen), 1.5 nM MgCl₂ (Invitrogen), 300 nM dNTP mix (Invitrogen), 400 nM primer pair (IDT, Coralville, Iowa), and 0.1 U/μl AmpliTaq Gold DNA polymerase (Invitrogen). PCR reactions were performed by a Mastercycler epgradient thermocycler (Eppendorf North America, Westbury, N.Y.). The reaction protocol was: 94° C., 2 min; 40 cycles of 94° C., 20 sec, 58° C., 15 see, 72° C., 42 sec; followed by 72° C., 30 sec. The oligos used as PCR primers are indicated in Example 11.

Antibodies and Reagents

Primary antibodies: Mouse anti-human heparanase monoclonal antibody was obtained from Cedarlane Laboratories (Burlington, N.C.); a pan-cytokeratin antibody for CK5/8/18 (sc-53262) from Santa Cruz Biotechnology Inc. (Santa Cruz, Calif.), and the pan-cytokeratin AE1 antibody was purchased from Millipore (Billerica, Mass.). Other primary antibodies were purchased from Cell Signaling Technology (Danvers, Mass.). Secondary antibodies: Alexa Fluor 546 goat anti-mouse IgG [H+L] and Alexa Fluor 488 goat anti-rabbit IgG [H+L] were purchased from Invitrogen (Carlsbad, Calif.); goat anti-rabbit IgG [H+L]-HRP and goat anti-mouse IgG [H+L]-HRP were purchased from Santa Cruz Biotechnology (Santa Cruz. CA): biotinylated universal anti-rabbit/mouse IgG [H+L] was purchased from Vector Laboratories (Burlingame, Calif.).

Western Blotting

Cells were serum-starved overnight (16 hr), then cell lysates were collected and examined for specific proteins by Western blot analyses as previously reported (Zhang L X, Sullivan P S, Goodman et al. 2011).

Immunofluorescence (IF) and Immunohistochemistry (IHC)

CTCs were grown on coverslips in 12-well plates and serum-starved for 16 hrs before indicated treatments. For IHC assays, cells were fixed with 4% formaldehyde in PBS, permeabilized with 0.1% Triton X-100, and blocked in 10% normal goat serum followed by incubation overnight (16 hrs) at 4° C. with the specific primary antibody (1:50-1:100 dilution) followed by secondary antibody (1:200-1:400 dilution) incubation for 1 hr at room temperature (25° C.). Samples were processed as described in figure legends. Nuclei were counterstained with DAPI for IF. Stained cells were analyzed using confocal microscopy (LSM 510 model, Carl Zeiss Inc., Jena, Germany). Expression of proteins was examined by IHC using formalin-fixed, paraffin-embedded mouse tissue, anti-HPSE monoclonal antibody (Cedarlane Inc., Burlington, N.C.) was used at 1:2,000 to 1:3,000 dilution, and incubated overnight at 4° C., followed by incubation with biotinylated anti-mouse IgG (H+L) for 30 min at 25° C. per manufacturer's instructions. Staining was performed using a Vectastain ABC kit (Vector Laboratories, Burlingame, Calif.). Pathologists blinded to study groups reviewed the stained and coded sections. Staining was distributed through a 0-3+intensity scale: 0 corresponded to background staining; 1+ for weak staining; 2+ for moderate staining; 3+ for strongest staining.

FISH

Fluorescence in situ hybridization (FISH) analyses for CACCs was performed per previous report (Katz et al., 2010). Two distinct FISH probe sets were used to detect EGFR gene gains either by amplification or polysomy. The combination of probes consisted of a locus specific identifier (LSI) for EGFR/Cep7p12 and Cep 7 (ploidy). Glass slides with maximal cell retention were placed into cytospin funnels (120-220 mm² in diameter) containing 5.0×10⁵-1.0×10⁶ PBMCs. Slides were then spun at 170 g×3 min at room temperature (25° C.), and allowed to air-dry before staining procedures. Cytospin preparations of PBMCs were immersed in 2×SSC for 2 min at 73° C., then in a protease solution for 4 min at 37° C. Slides were subsequently washed in 1×PBS, fixed for 5 min in 1° formaldehyde at 27° C., and washed again. Subsequent one-minute exposures to 70%, 85%, and 100% ethanol were used to dehydrate slides. After the slides dried, FISH probes were added to the target area, and cover slips were mounted with rubber cement. Co-denaturation occurred when the slides were incubated for 5 min at 73° C., and the following hybridization period was 16 hr at 37° C. The next day, slides were placed in 0.4×SSC/0.3% NP-40 for 2 minutes at 73° C. Slides were removed from the wash solution, stained with 4′ 6-diamide-2-phenylindole dihydrochloride (DAPI), and cover-slipped. Samples were stored at −20° C. until they were imaged. Fluorescent signals were scanned using an automated system (BioView Duet-3™ platform. BioView, Ltd. Rehovot, Israel), the results were classified on a cell-by-cell basis, assessing fluorescent signals and cell morphology. Genetic abnormalities that were scored include gene gains and deletions, monosomy, and polysomy.

Cell Invasion Analyses

Chemoinvasion analyses were performed using corresponding cell invasion kits (Cell Biolabs Inc., San Diego, Calif.) per manufacturer's instructions and experimental conditions previously reported (Zhang L X, Sullivan P S, Goodman J C et al., 2011).

Experimental Animal Metastasis

Female athymic nude mice (nu/nu, 4-5 weeks old) were purchased from Harlan Sprague Dawley, Inc. (Indianapolis. IN), and maintained at the accredited animal facility of Baylor College of Medicine (BCM). All studies were conducted according to NIH animal use guidelines and a protocol approved by the BCM Animal Care Committee. CTC lines (n=20 mice per CTC line per treatment group) were injected either intracardiacally or tail vein into nude mice (0.5×10⁶ cells per mouse) after animals were anesthetized with isofluorane. Mice were euthanized by CO₂ asphyxiation when they showed signs of neurological impairment (usually 4-6 weeks), the whole lungs and brains removed, and fixed in Bouin's solution. Serial sections, obtained by cutting every 300 μm of tissue, were analyzed by H&E staining for presence of lung and brain metastatic lesions. The presence of metastasis was confirmed by pathologists blinded to experimental groups. Brain metastases were then counted under a Zeiss microscope (100×-400× magnification) outfitted with an objective that contained an ocular grid with a 0.8 mm² area. Micro-metastases were defined as lesions ≦50 μm². The ≧50 μm² metric for macro-metastasis definition represents the mouse equivalent of the proportion of a magnetic resonance imaging for detectable brain metastasis (5 mm) to the length of a human brain (McGowan et al., 2011). Three independent experiments were performed; data were pooled and analyzed for statistical significance.

Statistical Analyses

All data were analyzed using ANOVA or Student's t test, and represent the mean+S.D. of at least triplicate samples. A p value less than 0.05 was considered statistically significant. Statistical tests were performed with SAS statistical software (version 9.1; SAS Institute, Cary, N.C.).

### Example 11

### Exemplary RNA Embodiments

In embodiments of the invention, one may employ PCR to determine expression levels of one or more particular genes.

The inventors isolated total RNA from peripheral blood mononuclear cells (PBMC) using the RNeasy Plus Mini Kit with QIAshredder (QIAGEN. Valencia, Calif.) according to manufacturer's instructions. For each sample, 1 μg total RNA was digested with DNaseI (Invitrogen, Carlsbad, Calif.) as per manufacturer's instructions in a final volume of 11 μL The reverse transcriptase reaction was accomplished with a Super Script First Strand Synthesis kit (Invitrogen) consuming 4 μL, of the DNaseI digest reaction, which was immediately diluted 1:1 with ice cold RNase free water for a final volume of 80 μL. Each PCR reaction used 2 μL of first strand reaction. The final volume of each PCR reaction was 20 μL. The reaction mix had a final concentration of 1× Amp Gold buffer (Invitrogen), 1.5 nM MgCl₂(Invitrogen), 300 nM dNTP mix (Invitrogen), 400 nM primer pair (IDT, Coralville, Iowa), and 0.1 u/μL AmpliTaq Gold DNA polymerase (Invitrogen). The PCR reactions were performed in a Mastercycler epgradient (Eppendorf, Hauppauge, N.Y.). An example of a reaction protocol is as follows: 94° C. 2 min; 40 cycles of 94° C. 20 sec, 58° C. 15 sec, 72° C. 42 sec; followed by 72° C. 30 sec. The oligos used as PCR primers were: GAPDH, FP: TTC CAC CCA TGG CAA AT CC (SEQ ID NO: 1), RP: TGG CAG GTT TTT CTA GAC GG (SEQ ID NO:2), amplicon size: 611 bp; HPSE, FP: CTG GCA ATC TCA AGT CAA CC (SEQ ID NO:3), RP: TCC TAA CCA GAC CTT CTT GC (SEQ ID NO:4), amplicon size: 676 bp; NOTCH1 FP: GAA ACA ACT GCA AGA ACG GG (SEQ ID NO:5). RP: CTC ATT GAT CTT GTC CAG GC (SEQ ID NO:6), amplicon size: 746 bp; EPCAM, FP: GCT TTA TGA TCC TGA CTG CG (SEQ ID NO:7), RP: CAG CCT TCT CAT ACT TTG CC (SEQ ID NO:8), amplicon size: 623 bp; EGFR, FP: GAG GGC AAA TAC AGC TTT GG (SEQ ID NO:9), RP: GCT GTT TTC ACC TCT GTT GC (SEQ ID NO: 10), amplicon size: 651 bp; HER2, FP: AAT TAC AGA CTT CGG GGT GG (SEQ ID NO: 11), RP: GGC TGG TTC ACA TAT TCA GG (SEQ ID NO:12), amplicon size: 849 bp; CD45, FP: ACA GAT TTT GGG AGT CCA GG (SEQ ID NO:13). RP: GTA GAG AAC AAC AAG CAG GG (SEQ ID NO:14), amplicon size: 639 bp; VIMENTIN, FP: AGA ATA AGA TCC TGC TGG CC (SEQ ID NO: 15), RP: TAT TCA CGA AGG TGA CGA GC (SEQ ID NO: 16), amplicon size: 769 bp; TWIST, FP: ATA AGA GCC TCC AAG TCC GC (SEQ ID NO: 17), RP: GTA GAG GAA GTC GAT GTA CC (SEQ ID NO: 18), amplicon size: 826 bp; UPAR (detects splice variants 1, 2, and 3), FP: TCT TTC GCA AAA CGT CTG GG (SEQ ID NO: 19), RP: TTC TTC ACC TTC CTG GAT CC (SEQ ID NO:20), amplicon size: 584 bp; CAVEOLIN1 (pan) FP: AAG AAT TCC AGG GTA TGG CC (SEQ ID NO:21), RP: TGT CAC AGC ATA ACA GAC GG (SEQ ID NO:22), amplicon size: 799 bp, MUCIN1, FP: TAC CCA GAG AAG TTC AGT GC (SEQ ID NO:23), RP: TAC AAG TTG GCA GAA GTG GC (SEQ ID NO:24), amplicon size; 705 bp; KRT8, FP: CCC TCA ACA ACA AGT TTG CC (SEQ ID NO:25), RP: GCT CCT CAT ACT TGA TCT GG (SEQ ID NO:26), amplicon size: 576 bp; KRT18, FP: AGA AGG AGA CCA TGC AAA GC (SEQ ID NO:27), RP: GAT TTC TCA TGG AGT CCA GG (SEQ ID NO:28), amplicon size: 708 bp; KRT19, FP: CAA CGA GAA GCT AAC CAT GC (SEQ ID NO:29), RP: TGCAGCTCAATCTCAAGACC (SEQ ID NO:30), amplicon size: 649 bp; KRT20, FP: CCT CAA AAA GGA GCA TCA GG (SEQ ID NO:31), RP: AAA ACC TCA GCA CCA TCT CC (SEQ ID NO:32), amplicon size: 700 bp; CD24, FP: AGA GTA CTT CCA ACT CTG GG (SEQ ID NO:33). RP: AAA TCC AAA GCC TCA GGA GG (SEQ ID NO:34), amplicon size: 732 bp; CD44 (common to all but v8), FP: AGC TAG TGA TCA ACA GTG (GC (SEQ ID NO:35), RP: ATC AA.A GGA CTG ATC CAG GG (SEQ ID NO:36), amplicon size: 795 bp; CD44 (v8). FP: ACG TGG AGA AAA ATG GTC GC (SEQ ID NO:37), RP: AGA TCC ATG AGT GGT ATG GG (SEQ ID NO:38), amplicon size: 617 bp.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
Aktas B. Tewes M, Fehm M. et al. Stem cell and epithelial-mesenchymal transition markers are frequently overexpressed in circulating tumor cells of metastatic breast cancer patients. Breast Cancer Research. 2009. 11: R46 (doi: 10.1186/bcr2333).
Ameri K, Luong R, Zhang R, Powell A A, Montgomery K D. Espinosa I, Bouley D M, Harris A L, and Jeffrey S S. Circulating tumor cells demonstrate an altered response to hypoxia and an aggressive phenotype. Br J Cancer 2010; 102:561-9.
Blackwell K L, Pegram M D, Tan-Chiu E, et al. Single-agent lapatinib for HER2-overexpressing advanced or metastatic breast cancer that progressed on first- or second-line trastuzumab-containing regimens. Ann. Oncol. 2009. 20: 1026-1031.
Bolos V, Blanco M, Medina V, Aparicio G, Diaz-Prado S, Grande E. Notch signalling in cancer stem cells. Clin. Transl. Oncol. 2009. 11: 11-19.
Bos D P. Zhang X H-F, Nadal C, Shu W. Gomis R R, Nguyen D X. Minn A J, van de Vijer M J, Gerald W L, Foekens J A, Massague J Genes that mediate cancer metastasis to brain. Nature 2009. 459(7249): 1-8.
Bouras T, Pal B, Vaillant F, Harburg G, Asselin-Labat M L, Oakes S R, et al. Notch signaling regulates mammary stem cell function and luminal cell-fate commitment. Cell Stem Cell 2008. 3: 429-441.
Casu B. Vlodavsky I, Sanderson R D. Non-anticoagulant heparins and inhibition of cancer. Pathophysiol. Haemost. Thromb., 2008. 36: 195-203.
Chen J. Imanaka N, Chen J, Griffin J. Hypoxia potentiates Notch signaling in breast cancer leading to decreased E-cadherin expression and increased cell migration and invasion. Br. J. Cancer 2010. 102:351-360.
Cohen-Kaplan V, Dowek I, Naroditsky I, Vlodavski I, and Ilan N. Heparanase augments epidermal growth factor receptor phosphorylation: correlation with head and neck tumor progression. Cancer Res 2008; 68(24): 10077-85.
Cristofanilli M. Budd G T, Ellis M J, Stopeck A. Matera J. Miller M C. Reuben J M, Doyle G V, Allard W J, Terstappen L W, and Hayes D F. Circulating tumor cells, disease progression and survival in metastatic breast cancer. New Eng. J. Med. 2004: 351:781-91.
Dickson B C. Mulligan A M, Zhang H, Lockwood G, O'Malley F P, Egan S E, et al. High-level JAG 1 mRNA and protein predict poor outcome in breast cancer. Mod. Pathol. 2007. 20: 685-693.
Dominici M, Blanc Le K, Mueller I, Slaper-Cortenbach I, Marini F C. Krause D S, Deans R J, Keating A, Prockop D J, and Horwitz E M. Minimal criteria for defining multipotent mesenchymal stromal cells. The International Society for Cellular Therapy position statement. Cytotherapy 2006; 8(4): 315-7.
Dlontu G, Jackson K W, McNicholas E, Kawamura M J, Abdallah W M, Wicha M S. Role of Notch signaling in cell-fate determination of human mammary stem/progenitor cells. Breast Cancer Res. 2004. 6: R605-15.
Eichler A F, Chung E, Kodack D P, Jay S, Loeffler J S, Fukumura D, and Jain R K. The biology of brain metastases—translation to new therapies Nat. Rev. Clin. Oncol. 2011; 8: 344-56.
Farnie G, Clarke R B. Mammary stem cells and breast cancer—role of Notch signalling. Stem Cell Rev. 2003: 169-175.
Fehm T, Müller V, Aktas B. Janni W, Schneeweiss A, Stickeler E, Lattrich C, Löhberg C R, Solomayer E, Rack B, Riethdorf S, Klein C, Schindlbeck C, Brocker K, Kasimir-Bauer S. Wallwiener D, and Pantel K. HER2 status of circulating tumor cells in patients with metastatic breast cancer: a prospective, multicenter trial. Br Cancer Res and Treat 2010; 124(2):403-12.
Fonsatti E, Jekunen A P, Kairemo K J A, Coral S, Snellman M, Nicotra M R, Natali P G, Altomonte M, and Maio M. Endoglin is a suitable target for efficient imaging of solid tumors: in vivo evidence in a canine mammary carcinoma model. Clin Cancer Res 2000; 6: 2037-43.
Ginestier C, Hur M I, Charafe-Jauffret E, Monville F, Dutcher J. Brown M, Jacquemier J, Viens P, Kleer C G, Liu S, Schott A, Hayes D, Birnbaum D, Wicha M S, and Dontu G. ALDH1 is a marker of normal and malignant human mammary stem cells and a predictor of poor clinical outcome. Cell Stem Cell 2007: 1:555-67.
Gril B, Palmieri D, Bronder J L, Herring J M, Vega-Valle E, Feigenbaum L. Liewehr D J, Steinberg S M, Merino M J, Rubin S D, Steeg P S. Effect of lapatinib on the outgrowth of metastatic breast cancer cells to the brain. J. Natl. Cancer Inst. 2008. 100(15): 1092-1103.
Hafeez B B, Adhami V, Asim M, Siddiqui I A, Bhat K M, Zhong W, et al. Targeted knockdown of Notch1 Inhibits invasion of human prostate cancer cells concomittant with inhibition of matrix metalloproteinase and urokinase plasminogen activator. Clin. Cancer Res. 2009. 15: 452-459.
Harrell J C, Prat A. Parker J S. Fan C. He, X. Carey L, Anders C, Ewend M, and Perou C M. Genomic analysis identifies unique signatures predictive of brain, lung, and liver relapse. Breast Cancer Res Treat 2012; 132(2): 523-35; Epubl. Jun. 14, 2011.
Hayes D F, Cristofanilli M, Budd G T, et al. Circulating tumor cells at each follow-up time point during therapy of metastatic breast cancer patients predict progression-free and overall survival. Clin. Cancer Res. 2006. 12: 4218-4224.
Hicks D, Short S. Prescott N, et al. Breast cancers with brain metastasis are more likely to be estrogen receptor negative, express the basal cytokeratin CK5/6 and over-express Her-2 or EGFR. Am. J. Surg. Pathol. 2006. 30(9): 1097-1104.
Hirose I H. Ishii H, Mimori K, Ohta D. Ohkuma M, Tsujii H, Saito T, Sekimoto M. Doki Y, and Mori M. Notch pathway as candidate therapeutic target in Her2/Neu/ErbB2 receptor-negative breast tumors. Oncol Rep 2010; 1:35-43.
Hu X, Feng F, Wang Y, Wang L, He F, Dou G R, et al. Blockade of Notch signaling in tumor-bearing mice may lead to tumor regression, progression or metastasis, depending on tumor cell types. Neoplasia 2009. 11: 32-38.
Hughes D. How the NOTCH pathway contributes to the ability of osteosarcoma cells to metastasize. Cancer Treat. Res. 2010. 152: 479-496.
Ikediobi O N, Davies H, Bignell G, Edkins S, Stevens C, O'Meara S, Santarius T, Avis T, Barthorpe S, Brackenbury L, Buck G. Butler A. Clements J, Cole J, Dicks E, Forbes S, Gray K, Halliday K, Harrison R, Hills K, Hinton J, Hunter C, Jenkinson A, Jones D, Kosmidou V, Lugg R, Menzies A, Mironenko T, Parker A, Perry J, Raine K, Richardson D, Shepherd R, Small A, Smith R, Solomon H, Stephens P, Teague J, Tofts C, Varian J, Webb T, West S, Widaa S, Yates A, Reinhold W, Weinstein J N, Stratton M R, Futreal P A, and Wooster R. Mutation analysis of 24 known cancer genes in the NCI-60 cell line set. Mol. Cancer. Ther. 2006; 5(11): 2606-12.
Jiang F. Qiu Q, Khanna A, et al. Aldehyde dehydrogenase 1 is a tumor stem cell associated marker in lung cancer. Mol. Cancer. Res. 2009. 7(3): 330-338.
Joosse S A, Hannemann J. Spotter J, Bauche A. Andreas A. Muller V, and Pantel K. Changes in keratin expression during metastatic progression of breast cancer: Impact on the detection of circulating tumor cells. Clin Cancer Res 2012; 18(4): 993-1003.
Kaiser J. Cancer's circulation problem. Science 2010; 327:1072-4.
Kakarala M. Wicha M S. Cancer stem cells: implications for cancer treatment and prevention. Cancer J. 2007. 13: 271-275.
Katz R L, He W, Khanna A, Fernandez R L, Zaidi™, Krebs M, Caraway N P, Zhang H Z, Jiang F, Spitz M R. Blowers D P, Jimenez C A, Mehran R J, Swisher S O, Roth J A, Morris J S, Etzel C J, and El-Zein R. Genetically abnormal circulating cells in lung cancer patients: an antigen independent fluorescence in situ hybridization—based control study. Clin. Cancer Res. 2010; 16(15):3976-87.
Khanna A, Zaidi T, Fernandez R, et al. Circulating ALDH1 positive cells with chromosome 10/10q and EGFR abnormalities are novel biomarkers for monitoring cancer stem cells and correlate with ALDH1 positive cells of non-small cell lung cancer: An immunocytochemistry-FISH based study. Abstract 122, Cancer Cytopathology, Oct. 25, 2010.
Königsberg R. Obermayr E. Bises G, Pfeiler G, Gneist M. Wrba F, de Santis M, Zeillinger R, Hudec M, and Dittrich C. Detection of EpCAM positive and negative circulating tumor cells in metastatic breast cancer patients. Acta Oncol 2011; 50: 700-10.
Li X, Lewis M T, Huang J, Gutierrez C, Osborne K C, Wu M, Hilsenbeck S G, Pavlick A, Zhang X, Chamness G C, Wong H. Rosen J, and Chang J C. Intrisic resistance of tumorigenic breast cancer cells to chemotherapy. J Natl Cancer Inst. 2008; 100: 672-9.
Lu J, Steeg P S, Price J E, et al. Breast cancer metastasis: challenges and opportunities. Cancer Res. 2009. 69: 4951-4953.
Maheswaran S, Sequist L V. Nagrath S, Ulkus L, Brannigan B, Collura C V. Inserra E, Diederichs S, Iafrate A J, Bell D W, Digumarthy S, Muzikansky A, Irimia D, Settleman J, Tompkins R G, Lynch T J, Toner M, and Haber D A. Detection of mutations in EGFR in circulating lung-cancer cells. New Engl. J. Med 2008; 359, 366-377.
Marchetti D and Nicolson G L. Human heparanase: A molecular determinant of brain metastasis. Adv. Enz. Reg. 2001. 41: 343-359. PMCID: PMC 11384754.
Marchetti, D., Li J., Shen, R-J. Astrocytes contribute to the brain-metastatic specificity of melanoma cells by producing heparanase. Cancer Res. 2000. 60: 4767-4770. PMCID: PMC10987284.
Marrotta L and Polyak K. Cancer stem cells: A model in the making. Curr. Opin Genet. Dev. 2009. 19(10): 44-50.
Marusyk A and Polyak K. Tumor heterogeneity: Causes and consequences. Biochim. Biophys. Acta. 2010. 1805(1): 105-117.
McGowan P M, Simedrea C, Ribot E J, Foster P J, Palmieri D. Steeg P S, Allan A L, and Chambers A F. Notch1 inhibition alters the CD44hi/CD24lo population and reduces formation of brain metastases from breast cancer. Mol Cancer Res 2011: 9(7): 834-44.
Meerbrey K. Hu G. Kessler J, et al. The pINDUCER lentiviral toolkit for inducible RNA interference in vitro and in vivo. Proc. Natl. Acad. Sci. (USA) 2011. 108(9): 3665-3670.
Mego M, De Giorgi U, Dawood S, Wang X, Valero V. Andreopoulou E, Handy B, Ueno N T, Reuben J M, and Cristofanilli M. Characterization of metastatic breast cancer patients with nondetectable circulating tumor cells. Int J Cancer 2011; 129:417-23.
Mego M, Mani S A, and Cristofanilli M. Molecular mechanisms of metastasis in breast cancer clinical applications. Nat Rev Clin Oncol 2010; 7:693-701.
Naggi A. Casu B, Perez M, Torri G, et al. Modulation of the heparanase-inhibiting activity of heparin through the selective desulfation, graded N-acetylation, and glycol splitting. J. Biol. Chem. 2005. 280(13): 12103-12113.
Nagrath S, Sequist L V, Maheswaran S, Bell D W. Irimia D, Ulkus L, Smith M R, Kwak E L, Digumarthy S, Muzikansky A, Ryan P, Balis U J, Tompkins R G, Haber D A, and Toner M. Isolation of rare circulating tumor cells in cancer patients by microchips technology. Nature 2007. 450, 1235-1239.
Nam D, Jeon H. Kim S, Kim M H, Lee Y J, Lee M S, et al. Activation of Notch signaling in a xenograft model of brain metastasis. Clin Cancer Res 2008; 14: 4059-4066.
Neumeister V, Agarwal S, Bordeaux J, et al. In situ hybridization of putative cancer stem cells by Multiplexing ALDH1, CD44, and cytokeratin identifies breast cancer patients with poor prognosis. Am. J. Path. 2010. 176: 213-218.
Ostapkowicz A, Inai K, Smith L, Kreda S, Spychala J. Lipid rafts remodeling in estrogen receptor-negative breast cancer is reversed by histone deacetylase inhibitor. Mol Cancer Ther 2006; 5(2):238-45.
Palmieri D, Bronder J L, Herring J M, at al. Her-2 overexpression increases the metastatic outgrowth of breast cancer cells in the brain. Cancer Res. 2007. 67(9): 4190-4198.
Pannuti A, Foreman K, Rizzo P, Osipo C, Golde T, Osborne B, et al. Targeting Notch to target cancer stem cells. Clin. Cancer Res. 2010. 16: 3141-3152.
Pantel K, Brakenhoff R H, and Brandt B. Detection, clinical relevance and specific biological properties of disseminating tumor cells. Nat. Rev. Cancer 2008; 8:329-40.
Park S Y, Lee H I, Li H, Shipitisin M, Gelman R, Polyak K. Heterogeneity of stem cell-related markers according to tumor subtype and histologic stage of breast cancer. Clin. Cancer Res. 2010. 16(3):876-887.
Paterlini-Brechot P, and Benali L. Circulating tumor cells (CTCs) detection: clinical impact and future directions. Cancer Letters, 2007. 253: 180-204.
Pecot C V, Bischoff F Z, Mayer J A, Wong K L., Pham T, Bottsford-Miller J, Stone R L, Lin Y G, Jaladurgam P, Roh J W. Goodman B W, Merritt W M. Pircher T J. Mikolajczyk S D, Nick A M, Celestino J, Eng C, Ellis L M, Deavers M T, and Sood A K. A novel platform for detection of CK+ and CK− CTCs. Cancer Discovery 2011; 1(7): 580-6.
Polyak K and Weinberg R A. Transitions between epithelial and mesenchymal states: acquisition of Malignant and stem cell traits. Nat. Rev. Cancer. 2009. 9(4): 265-273.
Rhim A D, Mirek E T, Aiello N M, Maitra A, Bailey J M, McAllister F, Reichert M. Beatty G L, Rustgi A K, Vonderheide R H, Leach S D, and Stanger B Z. EMT and dissemination precede pancreatic tumor formation. Cell 2012; 148: 349-61.
Ridgway L. D., Wetzel M., Eptsein A, and Marchetti, D. Heparanase-induced, GEF-H1-mediated signaling in brain metastatic breast cancer cell extravasation and cytoskeletal dynamics. Mol. Cancer. Res., 2011, submitted.
Ridgway L. D., Wetzel M., Marchetti, D. Heparanase effects on proteoglycan-induced signaling in brain metastatic melanoma. Journ. Cell. Biochemistry. 2010. 111(5): 1299-1309 (selected as feature article).
Ridgway L D, Wetzel M, Epstein A, and Marchetti D. Heparanase-induced GEF-H1 signaling regulates the cytoskeletal dynamics of brain metastatic breast cancer cells. Mol Cancer Res. 2012; 10(6): 689-702.
Rimawi M F, Shetty P B, Weiss H L, Schiff R, Osborne K. Chamness G C, and Elledge R M. EGFR expression in breast cancer association with biologic phenotype and clinical outcomes. Cancer 2010; 116(5): 1234-46.
Ritchie J P, Ramani V C, Ren Y, et al. SST0001, a chemically modified heparin, inhibits myeloma growth and angiogenesis via disruption of the heparanase/syndecan-1 axis. Clin. Cancer Res. 2011, March 8, (E-publ. ahead of print).
SAS/STAT 9 User's Guide. SAS Institute. 2002.
Sieuwerts A M, Kraan J, Bolt J, et al. Anti-epithelial cell adhesion molecule antibodies and the detection of circulating normal-like breast tumor cells. J. Natl. Cancer Inst. 2009. 101: 61-66.
Smid M, Wang Y, Zhang Y, et al. Subtypes of breast cancer show preferential site of relapse. Cancer Res. 2008. 68(9): 3108-3114.
Stott S L, Lee R J. Nagrath S, Yu M, Miyamoto D T, Ulkus L, Inserra E J, Ulman M, Springer S, Nakamura Z, Moore A L, Tsukrov D I, Kempner M E, Dahl D M, Wu C L, Iafrate A J, Smith M R, Tompkins R G, Sequist L V, Toner M, Haber D A, and Maheswaran S. Isolation and characterization of circulating tumor cells from patients with localized and metastatic prostate cancer. Science Transl Med 2010; 2(25): 1-10.
Stylianou S, Clarke R B, Brennan K. Aberrant activation of Notch signaling in human breast cancer. Cancer Res. 2006. 66: 1517-1725.
Talmadge J E and Fidler I. AACR Centennial Series: The biology of cancer metastasis. Cancer Res 2010; 70(14): 5649-69.
Tarin D. Inappropriate gene expression in human cancer and its far-reaching biological and clinical significance. Cancer Metastasis Rev. 2012; 31(1-2): 21-39.
Vreys V and David G. Mammalian heparanase: What is the message?J. Cell. Mol. Med. 2007. 11(3): 427-452.
Zhang L, Sullivan P, Suyama J, Marchetti D. EGF-induced heparanase nucleolar localization augments DNA Topoisomerase I activity in brain metastatic breast cancer cells. Mol. Cancer. Res. 2010. 8(2): 278-290.
Zhang L X, Sullivan P S. Goodman J C. Gunaratne P H, and Marchetti D. MicroRNA-1258 suppresses breast cancer brain metastasis by targeting heparanase. Cancer Res 2011; 71(3): 645-54. Breaking advances—highlights from recent cancer literature. Cancer Res 2011; 71(6): 2025.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of identifying the presence of or risk for brain metastatic breast cancer in an individual, comprising the step of identifying from a sample from the individual circulating cells that are epithelial cell adhesion molecule (EpCAM) negative and that comprise expression of heparanase (HPSE) and/or Notch1.

2. The method of claim 1, wherein the cells further comprise one or more of the following markers:
a) HER2/neu;
b) EGFR;
c) uPAR;
d) ALDH1;
e) cytokeratins;
f) CD44^{high}/CD24_{low};
g) vimentin; and
h) CD45.

3. The method of claim 1, wherein the cells are circulating tumor cells (CTCs).

4. The method of claim 1, wherein the cells are peripheral blood mononuclear cells.

5. The method of claim 3, wherein the HPSE is localized to the nucleus or nucleolus of cells from the CTCs from the sample.

6. The method of claim 2, wherein the presence of the markers is determined by immunofluorescence, fluorescence in situ hybridization, flow cytometry, polymerase chain reaction, or a combination thereof.

7. The method of claim 1, wherein the method is employed in conjunction with another method for identifying brain metastatic breast cancer or breast cancer in an individual.

8. A method of identifying the presence of or risk for brain metastatic breast cancer in an individual, comprising the step of identifying from a sample from the individual circulating cells that are epithelial cell adhesion molecule (EpCAM) negative.

9. A method of treating an individual for brain metastatic breast cancer or delaying the onset of brain metastatic breast cancer in an individual, or preventing brain metastatic breast cancer in an individual or preventing metastasis of breast cancer in an individual or preventing breast cancer, comprising the step of providing an effective amount of a therapy to the individual when the individual has had identified the presence of circulating cells that are epithelial cell adhesion molecule (EpCAM) negative and that comprise expression of heparanase (HPSE) and/or Notch1 in a sample from the individual.

10. The method of claim 9, wherein the therapy is selected from the group consisting of surgery, radiation, immunotherapy, chemotherapy, hormone therapy, steroids, and a combination thereof.

11. The method of claim 9, wherein the cells further comprise one or more of the following markers:
a) HER2/neu;
b) EGFR;
c) uPAR;
d) ALDH1;
e) cytokeratins;
f) CD44^{high}/CD24_{low};
g) vimentin; and
h) CD45.


SEQUENCE LISTING (38 sequences)

SEQ ID NO: 1 - DNA, 20 bp, Artificial Sequence
  Synthetic Primer
ttccacccatggcaaattcc

SEQ ID NO: 2 - DNA, 20 bp, Artificial Sequence
  Synthetic Primer
tggcaggtttttctagacgg

SEQ ID NO: 3 - DNA, 20 bp, Artificial Sequence
  Synthetic Primer
ctggcaatctcaagtcaacc

SEQ ID NO: 4 - DNA, 20 bp, Artificial Sequence
  Synthetic Primer
tcctaaccagaccttcttgc

SEQ ID NO: 5 - DNA, 20 bp, Artificial Sequence
  Synthetic Primer
gaaacaactgcaagaacggg

SEQ ID NO: 6 - DNA, 20 bp, Artificial Sequence
  Synthetic Primer
ctcattgatcttgtccaggc

SEQ ID NO: 7 - DNA, 20 bp, Artificial Sequence
  Synthetic Primer
gctttatgatcctgactgcg

SEQ ID NO: 8 - DNA, 20 bp, Artificial Sequence
  Synthetic Primer
cagccttctcatactttgcc

SEQ ID NO: 9 - DNA, 20 bp, Artificial Sequence
  Synthetic Primer
gagggcaaatacagctttgg

SEQ ID NO: 10 - DNA, 20 bp, Artificial Sequence
  Synthetic Primer
gctgttttcacctctgttgc

SEQ ID NO: 11 - DNA, 20 bp, Artificial Sequence
  Synthetic Primer
aattacagacttcggggtgg

SEQ ID NO: 12 - DNA, 20 bp, Artificial Sequence
  Synthetic Primer
ggctggttcacatattcagg

SEQ ID NO: 13 - DNA, 20 bp, Artificial Sequence
  Synthetic Primer
acagattttgggagtccagg

SEQ ID NO: 14 - DNA, 20 bp, Artificial Sequence
  Synthetic Primer
gtagagaacaacaagcaggg

SEQ ID NO: 15 - DNA, 20 bp, Artificial Sequence
  Synthetic Primer
agaataagatcctgctggcc

SEQ ID NO: 16 - DNA, 20 bp, Artificial Sequence
  Synthetic Primer
tattcacgaaggtgacgagc

SEQ ID NO: 17 - DNA, 20 bp, Artificial Sequence
  Synthetic Primer
ataagagcctccaagtctgc

SEQ ID NO: 18 - DNA, 20 bp, Artificial Sequence
  Synthetic Primer
gtagaggaagtcgatgtacc

SEQ ID NO: 19 - DNA, 20 bp, Artificial Sequence
  Synthetic Primer
tctttcgcaaaacgtctggg

SEQ ID NO: 20 - DNA, 20 bp, Artificial Sequence
  Synthetic Primer
ttcttcaccttcctggatcc

SEQ ID NO: 21 - DNA, 20 bp, Artificial Sequence
  Synthetic Primer
aagaattccagggtatggcc

SEQ ID NO: 22 - DNA, 20 bp, Artificial Sequence
  Synthetic Primer
tgtcacagcataacagacgg

SEQ ID NO: 23 - DNA, 20 bp, Artificial Sequence
  Synthetic Primer
tacccagagaagttcagtgc

SEQ ID NO: 24 - DNA, 20 bp, Artificial Sequence
  Synthetic Primer
tacaagttggcagaagtggc

SEQ ID NO: 25 - DNA, 20 bp, Artificial Sequence
  Synthetic Primer
ccctcaacaacaagtttgcc

SEQ ID NO: 26 - DNA, 20 bp, Artificial Sequence
  Synthetic Primer
gctcctcatacttgatctgg

SEQ ID NO: 27 - DNA, 20 bp, Artificial Sequence
  Synthetic Primer
agaaggagaccatgcaaagc

SEQ ID NO: 28 - DNA, 20 bp, Artificial Sequence
  Synthetic Primer
gatttctcatggagtccagg

SEQ ID NO: 29 - DNA, 20 bp, Artificial Sequence
  Synthetic Primer
caacgagaagctaaccatgc

SEQ ID NO: 30 - DNA, 20 bp, Artificial Sequence
  Synthetic Primer
tgcagctcaatctcaagacc

SEQ ID NO: 31 - DNA, 20 bp, Artificial Sequence
  Synthetic Primer
cctcaaaaaggagcatcagg

SEQ ID NO: 32 - DNA, 20 bp, Artificial Sequence
  Synthetic Primer
aaaacctcagcaccatctcc

SEQ ID NO: 33 - DNA, 20 bp, Artificial Sequence
  Synthetic Primer
agagtacttccaactctggg

SEQ ID NO: 34 - DNA, 20 bp, Artificial Sequence
  Synthetic Primer
aaatccaaagcctcaggagg

SEQ ID NO: 35 - DNA, 20 bp, Artificial Sequence
  Synthetic Primer
agctagtgatcaacagtggc

SEQ ID NO: 36 - DNA, 20 bp, Artificial Sequence
  Synthetic Primer
atcaaaggactgatccaggg

SEQ ID NO: 37 - DNA, 20 bp, Artificial Sequence
  Synthetic Primer
acgtggagaaaaatggtcgc

SEQ ID NO: 38 - DNA, 20 bp, Artificial Sequence
  Synthetic Primer
agatccatgagtggtatggg